(19) United States
(12) Patent Application Publication
(10) Pub. No.: US 2018/0256855 A1
(43) Pub. Date: Sep. 13, 2018

(54) CATHETER ASSEMBLY AND METHOD FOR MANUFACTURING SAME

(71) Applicants: Terumo Kabushiki Kaisha, Tokyo (JP); Mitsubishi Pencil Company, Limited, Tokyo (JP)

(72) Inventors: Yuya TERASAWA, Nakakoma-gun, Yamanashi (JP); Hisami TAMANO, Fujioka-shi, Gunma (JP); Mitsuhiro KAWABATA, Fujioka-shi, Gunma (JP); Satoru SUMIYOSHI, Fujioka-shi, Gunma (JP); Makoto TAMURA, Fujioka-shi, Gunma (JP); Yusuke KYOGOKU, Fujioka-shi, Gunma (JP)

(73) Assignees: Terumo Kabushiki Kaisha, Tokyo (JP); Mitsubishi Pencil Company, Limited, Tokyo (JP)

(21) Appl. No.: 15/532,987

(22) PCT Filed: Sep. 10, 2015
(86) PCT No.: PCT/JP2015/075681

§ 371 Date: Jun. 2, 2017

(30) Foreign Application Priority Data
Dec. 4, 2014 (JP) 2014-246146

(51) Int. Cl. A61M 25/06; A61M 5/158

(52) U.S. Cl. CPC A61M 25/0625; A61B 2017/347; A61M 25/0606; A61M 5/158

(57) ABSTRACT

The needle protection cover of a catheter assembly is provided with: an inner cylinder; an outer cylinder capable of being axially displaced relative to the inner cylinder; a blocking body disposed in the inner cylinder in a displaceable manner; and a lock mechanism for restraining the blocking body at the position where the blocking body blocks an inner needle passage. As an inner needle is pulled out, the blocking body is pressed by contact sections formed on the outer cylinder, thereby being moved from an initial position to a blocking position, and the blocking body is held at the blocking position by the lock mechanism.

### TECHNICAL FIELD

The present invention relates to a catheter assembly, which punctures and remains indwelling in a blood vessel, for example, when performing an infusion on a patient, as well as to a manufacturing method for such a catheter assembly.

### BACKGROUND ART

Conventionally, when an infusion is carried out on a patient, for example, a catheter assembly has been used. This type of catheter assembly is equipped with a hollow catheter, a catheter hub that is fixed to a proximal end of the catheter, an inner needle that is inserted into the catheter and has a sharp tip at a distal end portion thereof, and an inner needle hub that is fixed to a proximal end of the inner needle. In the event that the catheter assembly is used to perform an infusion on a patient, the catheter together with the inner needle punctures a blood vessel of a patient, and after puncturing, the inner needle is withdrawn from the catheter while the catheter remains in a punctured condition in the patient. Thereafter, a connector, which is provided on a distal end of an infusion tube, is connected to the proximal end of the catheter hub, and an infusion solution is supplied into the patient's blood vessel through the infusion tube, the catheter hub, and the catheter.

Incidentally, during use of this type of catheter assembly, after the inner needle is withdrawn from the catheter, for preventing the inner needle that has a sharp tip from being touched inadvertently by the user, a catheter assembly has been proposed which is equipped with a safety mechanism with which the inner needle is covered following withdrawal thereof (for example, see Japanese Laid-Open Patent Publication No. 2002-126080).

The safety mechanism of the conventional catheter assembly is constituted such that, for example, a shutter member made from a metallic elastic member is arranged in the interior of a cover tube having an inner needle passage, and when the inner needle is withdrawn, the shutter member undergoes expansion due to a restoring force thereof when the inner needle is withdrawn, whereby the inner needle does not protrude out from the distal end of the cover tube.

### SUMMARY OF INVENTION

The present invention has been devised in relation to the above-described conventional technology, and has the object of providing a catheter assembly as well as a manufacturing method therefor, in which, without using a shutter member made of an elastic member, it is possible to block an inner needle passage accompanying a withdrawal operation of the inner needle, and to prevent the inner needle from protruding out to the exterior, as well as to maintain a state in which the inner needle passage is shielded.

To accomplish the aforementioned object, a catheter assembly according to the present invention comprises an inner needle including a sharp tip at a distal end thereof, a catheter through which the inner needle is inserted, a catheter hub connected to a proximal end part of the catheter, and a needle protective cover configured to cover at least the tip of the inner needle when the inner needle is withdrawn. The needle protective cover includes an inner tube including an inner needle passage penetrating in an axial direction, and which is detachably connected to the catheter hub, an outer tube inside of which the inner tube is arranged, and which is configured to be relatively displaceable in the axial direction with respect to the inner tube, a block body accommodated in the inner tube, and configured to be displaceable from an initial position at which the inner needle passage is not shielded to a shielding position at which the inner needle passage is shielded, and a lock mechanism configured to restrain the block body at the shielding position. The lock mechanism includes a latching member provided in the inner tube and configured to be elastically displaceable in interior of the inner tube, and a latched member disposed on the block body and configured to be capable of engagement with the latching member. When an operation to withdraw the inner needle is performed, accompanying a backward movement of the outer tube with respect to the inner tube, the block body is configured to be pressed by an abutting member formed in the outer tube to move from the initial position to the shielding position, the block body is configured to be retained at the shielding position by the lock mechanism.

According to the catheter assembly which is constructed in the above manner, when the operation to withdraw the inner needle is performed, the block body, which is arranged in the interior of the inner tube, is pressed by the abutting member provided in the outer tube, whereby the block body moves from the initial position to the shielding position. Thus, it is possible to shield the inner needle passage, and to prevent the needle from protruding to the exterior of the inner tube. Further, after the block body has moved to the shielding position, since movement of the block body to the initial position is prevented by the lock mechanism, protrusion of the inner needle to the exterior can more effectively be prevented. Furthermore, from the fact that the latching member of the lock mechanism is constituted to be elastically displaceable in the interior of the inner tube, and since it is easily deformed when the latched member overcomes the latching member, an increase in the operating force required to perform the withdrawal operation of the inner needle, which is caused by resistance to activation of the lock mechanism, can be suppressed, and can effectively be suppressed.

In the above-described catheter assembly, the latching member may be a latching tab supported in a cantilevered fashion by an inner surface of the inner tube.

In accordance with this configuration, since the latching member is easily deformed, resistance to activation of the lock mechanism can be effectively reduced.

In the above-described catheter assembly, the latching tab may be configured to extend in a direction from the initial position toward the shielding position of the block body.

In accordance with this configuration, since the latching member is easily deformed accompanying movement of the block body, resistance to activation of the lock mechanism can more effectively be suppressed.

In the above-described catheter assembly, on the latching tab, on a portion thereof that undergoes sliding contact with the block body when the block body is displaced from the initial position to the shielding position, an inclined guide is formed, which deviates to an inner side of the inner tube as the inclined guide is positioned from a supported end side toward a free end side of the latching tab.

In accordance with this configuration, under a guiding action of the inclined guide, displacement takes place smoothly between the block body and the latching tab, and resistance to activation of the lock mechanism can more effectively be suppressed.

In the above-described catheter assembly, the latching member may be a beam supported at both ends thereof by inner surfaces of the inner tube.

In accordance with this configuration as well, by the beam that is flexed elastically accompanying displacement of the block body, while suppressing resistance to activation of the lock mechanism, it is possible to reliably maintain the state in which the block body shields the inner needle passage.

In the above-described catheter assembly, the abutting member may include a first inclined surface which is inclined with respect to a direction of relative movement between the outer tube and the inner tube, and the block body may include a second inclined surface which faces toward the first inclined surface in a state of being positioned at the initial position.

In accordance with this configuration, when the abutting member presses the block body, because the inclined surfaces contact and slide against each other, the block body can be made to move stably and smoothly.

In the above-described catheter assembly, the first inclined surface and the second inclined surface may be arranged in plurality, respectively, while being separated in a widthwise direction of the block body.

In accordance with this configuration, movement of the block body can be realized in a more stable manner.

In the above-described catheter assembly, the block body may include a protrusion which protrudes in the widthwise direction and is pressed by the abutting member, an elastic piece, which is elastically deformable in the widthwise direction of the block body, may be formed on the outer tube, and the abutting member may be formed on the elastic piece.

In accordance with this configuration, in an assembly process of the catheter assembly, when the inner tube is inserted into the outer tube, the elastic piece is pressed by the block body and is elastically deformed toward an outer side. Therefore, the inner tube can be smoothly inserted into the outer tube without the inner tube and the outer tube becoming damaged.

The present invention further relates to a method for manufacturing the aforementioned catheter assembly. More specifically, a manufacturing method for a catheter assembly is characterized by a block body arranging step of arranging the block body in the initial position inside the inner tube, and an inner tube insertion step of, after the block body arranging step, inserting the inner tube into the outer tube up to an insertion completion position, wherein, in the inner tube insertion step, accompanying relative displacement of the block body with respect to the outer tube, the protrusion of the block body presses the elastic piece formed on the outer tube outwardly, and elastically displaces the elastic piece, whereby the protrusion overcomes the elastic piece.

In accordance with such a manufacturing method for the catheter assembly, in an assembly process of the catheter assembly, when the inner tube is inserted into the outer tube, the protrusion of the block body presses the elastic piece outwardly and overcomes the elastic piece. Therefore, the inner tube can be smoothly inserted into the outer tube without the inner tube and the outer tube becoming damaged.

In the above-described manufacturing method for the catheter assembly, the catheter assembly may include an inner needle hub connected to a proximal end part of the inner needle, the needle protective cover may include an adjoining tube in which the outer tube is slidably inserted, and which is slidably inserted in the inner needle hub, a needle fixing portion configured to retain the proximal end part of the inner needle may be formed integrally in the inner needle hub, and the catheter assembly may comprise a stopper configured to prevent the adjoining tube from being pulled out in a distal end direction from the inner needle hub. The manufacturing method may further include an adjoining tube insertion step of inserting the adjoining tube into the inner needle hub through a distal end opening of the inner needle hub, a stopper attachment step of, after the adjoining tube insertion step, attaching the stopper to the inner needle hub through the distal end opening of the inner needle hub, an outer tube insertion step of, after the stopper attachment step, inserting the outer tube into the adjoining tube through the distal end opening of the inner needle hub, and an inner tube preliminary insertion step of, after the outer tube insertion step and the block body arranging step, inserting the inner tube into the outer tube up to a predetermined preliminary fixing position, through a distal end opening of the outer tube.

In this manner, since the adjoining tube, the stopper, the outer tube, and the inner tube (the inner tube assembled together with the block body) are assembled sequentially in one direction with respect to the inner needle hub from the distal end side toward the proximal end side of the inner needle hub, ease of assembly can be enhanced. Further, since it is possible to adopt an assembly method in which components thereof can be assembled by dropping other components with reference to the inner needle hub, automated assembly by a robot can be easily realized.

According to the catheter assembly and the manufacturing method therefor of the present invention, it is possible to block the inner needle passage accompanying a withdrawal operation of the inner needle, and to prevent the inner needle from protruding out to the exterior, as well as to maintain a state in which the inner needle passage is shielded.

### BRIEF DESCRIPTION OF DRAWINGS

FIG. 1 is a perspective view showing a catheter assembly according to an embodiment of the present invention;

FIG. 2 is an exploded perspective view of the catheter assembly shown in FIG. 1;

FIG. 3 is a perspective cross-sectional view of the catheter assembly taken along line III-III of FIG. 1;

FIG. 4 is a perspective cross-sectional view of an inner tube and a peripheral location of the catheter assembly shown in FIG. 1;

FIG. 5A is a plan view of an inner tube, FIG. 5B is a cross-sectional view of the inner tube taken along line VB-VB of FIG. 5A, and FIG. 5C is a cross-sectional view of the inner tube taken along line VC-VC of FIG. 5A;

FIG. 6A is a perspective view shown from a front side of a block body, and FIG. 6B is a perspective view shown from a rear side of the block body;

FIG. 7A is a perspective cross-sectional view of an outer tube, FIG. 7B is a cross-sectional view of the outer tube taken along line VIIB-VIIB of FIG. 7A, and FIG. 7C is a cross-sectional view of the outer tube taken along line VIIC-VIIC of FIG. 7A;

FIG. 8 is a cross-sectional view showing a state in which a needle protective cover is extended maximally accompanying a withdrawal operation of an inner needle;

FIG. 9A is a perspective cross-sectional view showing a mutual positional relationship between the inner tube, the outer tube, and the block body immediately after the outer tube has started to move with respect to the inner tube, FIG. 9B is a cross-sectional view showing an inclined surface of the outer tube and an inclined surface of the block body at the same point in time as in FIG. 9A, and FIG. 9C is a cross-sectional view showing a relationship between latching tabs of the inner tube and the claw parts of the block body at the same point in time as in FIG. 9A;

FIG. 10A is a perspective cross-sectional view showing a mutual positional relationship between the inner tube, the outer tube, and the block body at a time that the outer tube has moved further with respect to the inner tube from the state shown in FIG. 9A, FIG. 10B is a cross-sectional view showing an inclined surface of the outer tube and an inclined surface of the block body at the same point in time as in FIG. 10A, and FIG. 10C is a cross-sectional view showing a relationship between latching tabs of the inner tube and the claw parts of the block body at the same point in time as in FIG. 10A;

FIG. 11A is a perspective cross-sectional view showing a mutual positional relationship between the inner tube, the outer tube, and the block body at a time that the outer tube has moved further with respect to the inner tube from the state shown in FIG. 10A, FIG. 11B is a cross-sectional view showing an inclined surface of the outer tube and an inclined surface of the block body at the same point in time as in FIG. 11A, and FIG. 11C is a cross-sectional view showing a relationship between latching tabs of the inner tube and the claw parts of the block body at the same point in time as in FIG. 11A;

FIG. 12A is a perspective cross-sectional view showing a mutual positional relationship between the inner tube, the outer tube, and the block body at a time that the outer tube has moved further with respect to the inner tube from the state shown in FIG. 11A, FIG. 12B is a cross-sectional view showing an inclined surface of the outer tube and an inclined surface of the block body at the same point in time as in FIG. 12A, and FIG. 12C is a cross-sectional view showing a relationship between latching tabs of the inner tube and the claw parts of the block body at the same point in time as in FIG. 12A;

FIG. 13A is a perspective cross-sectional view showing a mutual positional relationship between the inner tube, the outer tube, and the block body at a time that the outer tube has moved further with respect to the inner tube from the state shown in FIG. 12A, FIG. 13B is a cross-sectional view showing an inclined surface of the outer tube and an inclined surface of the block body at the same point in time as in FIG. 13A, and FIG. 13C is a cross-sectional view showing a relationship between latching tabs of the inner tube and the claw parts of the block body at the same point in time as in FIG. 13A;

FIG. 14 is a perspective view showing a state in which a catheter hub and an inner tube are separated from each other;

FIG. 15A is a cross-sectional view of a cover and hub assembly, and FIG. 15B is a cross-sectional view showing a state, in a manufacturing process of the catheter assembly, in which the inner needle is fixed in an inner needle hub;

FIG. 16A is a cross-sectional view showing a state, in the manufacturing process of the catheter assembly, in which the catheter hub is mounted in a distal end part of the inner tube, and FIG. 16B is a cross-sectional view showing a state in which the catheter hub is pressed in slightly toward the side of the inner needle hub from the state shown in FIG. 16A;

FIG. 17A is a cross-sectional view showing a state, in the manufacturing process of the catheter assembly, in which the catheter hub is pressed in further toward the side of the inner needle hub from the state shown in FIG. 16B, and FIG. 17B is a bottom view of the catheter assembly in the state shown in FIG. 17A;

FIG. 18 is a perspective cross-sectional view of the catheter assembly in a state upon completion of assembly thereof;

FIG. 19A is a plan view of an inner tube having beams therein, FIG. 19B is a cross-sectional view of the inner tube taken along line XIXB-XIXB of FIG. 19A, and FIG. 19C is a cross-sectional view of the inner tube taken along line XIXC-XIXC of FIG. 19A;

FIG. 20A is a perspective cross-sectional view showing a mutual positional relationship between the inner tube, the outer tube, and the block body immediately after the outer tube has started to move with respect to the inner tube, FIG. 20B is a cross-sectional view showing an inclined surface of the outer tube and an inclined surface of the block body at the same point in time as in FIG. 20A, and FIG. 20C is a cross-sectional view showing a relationship between the beams of the inner tube and the claw parts of the block body at the same point in time as in FIG. 20A;

FIG. 21A is a perspective cross-sectional view showing a mutual positional relationship between the inner tube, the outer tube, and the block body at a time that the outer tube has moved further with respect to the inner tube from the state shown in FIG. 20A, FIG. 21B is a cross-sectional view showing an inclined surface of the outer tube and an inclined surface of the block body at the same point in time as in FIG. 21A, and FIG. 21C is a cross-sectional view showing a relationship between the beams of the inner tube and the claw parts of the block body at the same point in time as in FIG. 21A;

FIG. 22A is a perspective cross-sectional view showing a mutual positional relationship between the inner tube, the outer tube, and the block body at a time that the outer tube has moved further with respect to the inner tube from the state shown in FIG. 21A, FIG. 22B is a cross-sectional view showing an inclined surface of the outer tube and an inclined surface of the block body at the same point in time as in FIG. 22A, and FIG. 22C is a cross-sectional view showing a relationship between the beams of the inner tube and the claw parts of the block body at the same point in time as in FIG. 22A;

FIG. 23A is a perspective cross-sectional view showing a mutual positional relationship between the inner tube, the outer tube, and the block body at a time that the outer tube has moved further with respect to the inner tube from the state shown in FIG. 22A, FIG. 23B is a cross-sectional view showing an inclined surface of the outer tube and an inclined surface of the block body at the same point in time as in FIG. 23A, and FIG. 23C is a cross-sectional view showing a relationship between the beams of the inner tube and the claw parts of the block body at the same point in time as in FIG. 23A; and

FIG. 24A is a perspective cross-sectional view showing a mutual positional relationship between the inner tube, the outer tube, and the block body at a time that the outer tube has moved further with respect to the inner tube from the state shown in FIG. 23A, FIG. 24B is a cross-sectional view showing an inclined surface of the outer tube and an inclined surface of the block body at the same point in time as in FIG. 24A, and FIG. 24C is a cross-sectional view showing a relationship between the beams of the inner tube and the claw parts of the block body at the same point in time as in FIG. 24A.

### DESCRIPTION OF EMBODIMENTS

A preferred embodiment of a catheter assembly according to the present invention, as well as a manufacturing method therefore, will be described in detail below with reference to the accompanying drawings. In the respective drawings relating to the catheter assembly, the X direction indicates an axial direction of the catheter assembly and constituent elements thereof, the X1 direction indicates a distal end direction, and the X2 direction indicates a proximal end direction. Further, the Y direction indicates a left and right horizontal direction (widthwise direction), and the Z direction indicates a vertical direction.

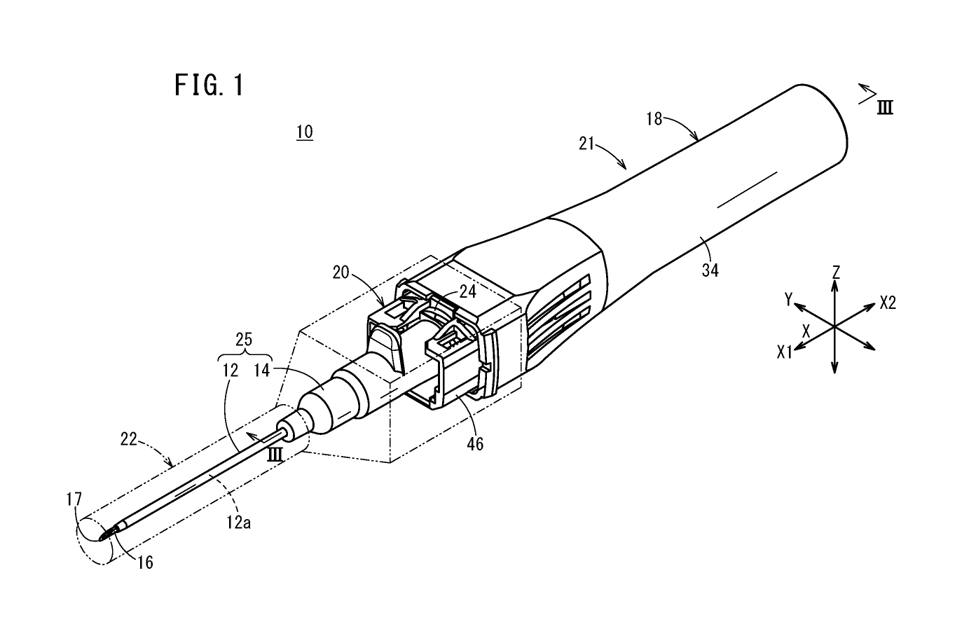

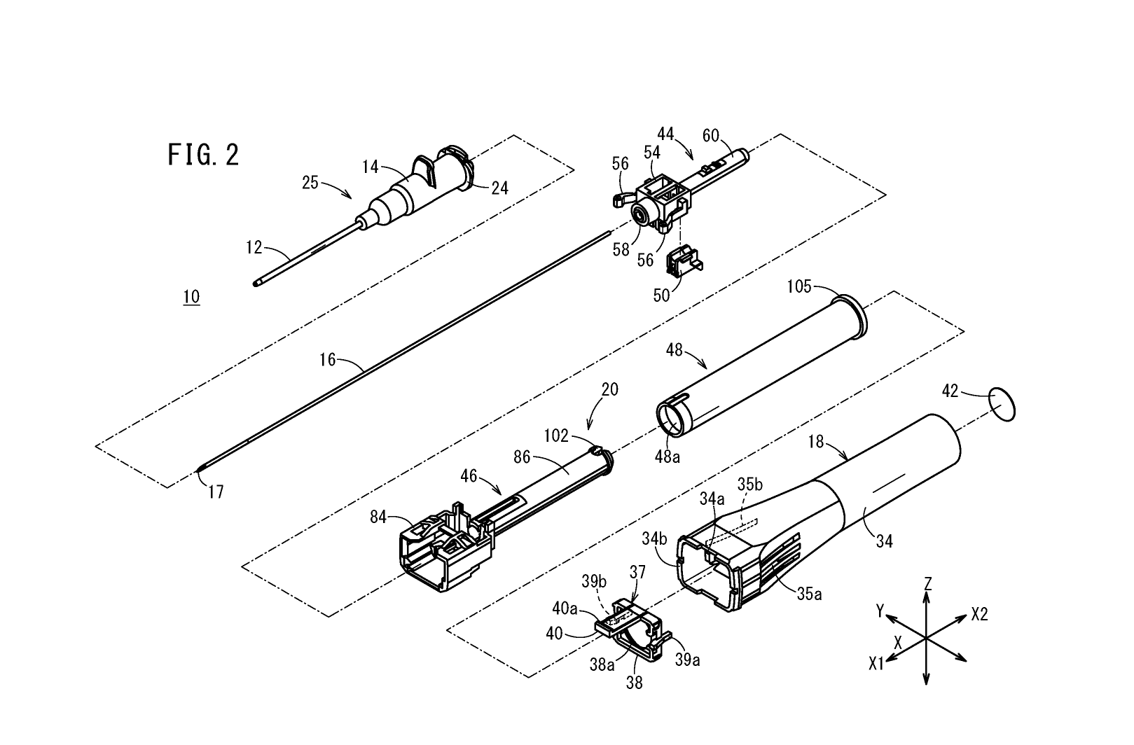

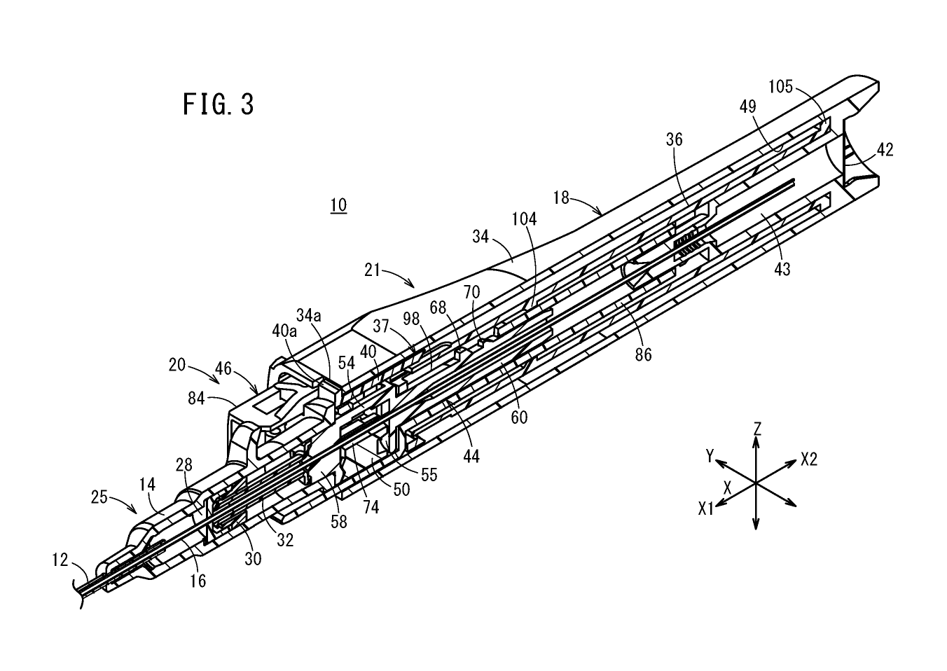

FIG. 1 is a perspective view of a catheter assembly 10 according to an embodiment of the present invention. FIG. 2 is an exploded perspective view of the catheter assembly 10. FIG. 3 is a perspective view showing in cross section the catheter assembly 10 taken along line III-III of FIG. 1.

As shown in FIGS. 1 and 2, the catheter assembly 10 is equipped with a tubular catheter 12, a catheter hub 14 that is connected to a proximal end side of the catheter 12, a tubular inner needle 16 having a sharp tip 17 on a distal end thereof and which is capable of being inserted through the interior of the catheter 12, an inner needle hub 18 connected to a proximal end side of the inner needle 16, and a needle protective cover 20 that covers at least the tip 17 of the inner needle 16 when the inner needle 16 is retracted.

Until the catheter assembly 10 is used, a protector 22, which provides covering from the tip 17 of the inner needle 16 up to a distal end part of the needle protective cover 20, is attached to a distal end part of the inner needle hub 18. The catheter assembly 10 is used in the following manner, which will be described in outline below.

A user (a doctor or medical technician, etc.) performs an operation to grip the inner needle hub 18 of the catheter assembly 10, whereby the distal end part thereof punctures and is inserted into a blood vessel of a patient. In an initial condition prior to use of the catheter assembly 10 (before puncturing the patient), the inner needle 16 is inserted through the catheter 12 in the form of a double tube structure, and the inner needle 16 projects a predetermined length from the distal end of the catheter 12. Further, in the initial condition of the catheter assembly 10, the proximal end side of the catheter hub 14 and the distal end side of the inner needle hub 18 are connected through the needle protective cover 20.

After the protector 22 has been removed from the catheter assembly 10, the catheter 12 and the inner needle 16 that make up the double tube structure are inserted together into the blood vessel of the patient. After puncturing the patient, in a condition in which the position of the catheter 12 is maintained, by retracting the inner needle hub 18 in the direction of the proximal end, when the needle protective cover 20 is made to separate away from the catheter hub 14, the inner needle 16 that is connected to the inner needle hub 18 also is pulled out integrally therewith, and is separated from the catheter 12 and the catheter hub 14. As a result, a state is brought about in which only the catheter 12 and the catheter hub 14 are left indwelling on the side of the patient.

When the inner needle 16 is withdrawn from the catheter 12, the inner needle 16 becomes accommodated inside the needle protective cover 20 due to the needle protective cover 20 extending to the distal end side of the inner needle hub 18 with respect to the inner needle hub 18. Consequently, exposure of the inner needle 16 to the exterior is prevented. After the inner needle 16 has been withdrawn from the catheter 12, a non-illustrated connector of an infusion tube is connected to the proximal end side of the catheter hub 14, whereby supply of an infusion agent (medicinal solution) is carried out from the infusion tube to the patient.

Below, the constitution of the catheter assembly 10 will be described in greater detail.

The catheter assembly 10 is constituted as a single assembly, in which the double tube structure of the catheter 12 and the inner needle 16, the catheter hub 14, the needle protective cover 20, and the inner needle hub 18 are combined and are capable of being handled integrally.

The catheter 12 in the catheter assembly 10 is a flexible and narrow diameter tubular member formed with a predetermined length. In the interior of the catheter 12, a lumen 12a is formed to extend and penetrate through the catheter 12 in the axial direction. The inner diameter of the lumen 12a is set to a size that enables the inner needle 16 to be inserted through the lumen 12a.

As the material composing the catheter 12, a resin, particularly, a soft resin material is preferred. In this case, for example, a fluororesin such as polytetrafluoroethylene (PTFE), ethylene-tetrafluoroethylene copolymer (ETFE), perfluoroalkoxy fluororesin (PFA), an olefin resin such as polyethylene and polypropylene or a mixture thereof, polyurethane, polyester, polyamide, polyether nylon resin, and a mixture of the olefin resin and ethylene-vinyl acetate copolymer may be used. The catheter 12 may be constituted from a transparent resin material, so that all or a portion of the interior is visible.

The catheter hub 14 is connected in a fixed manner to the proximal end of the catheter 12. The catheter hub 14 of the illustrated example is formed with a tapering tubular shape. The distal end part of the catheter hub 14 and the proximal end part of the catheter 12 are fixed together mutually in a liquid-tight state. A flange 24, which projects outwardly and extends in a circumferential direction, is provided on the proximal end of the catheter hub 14. Hereinafter, a coupling body of the catheter 12 and the catheter hub 14 will be referred to as a “catheter member 25”.

When the catheter assembly 10 is used, the catheter hub 14 is exposed on the patient's skin in a state in which the catheter 12 has pierced into the blood vessel, and is pasted and held in place on the skin by tape or the like. The catheter hub 14 preferably is constituted from a material that is more rigid than the catheter 12. The constituent material of the catheter hub 14 is not limited to any particular material, however, a thermoplastic resin material, for example, polypropylene, polycarbonate, polyamide, polysulfone, polyarylate, methacrylate-butylene-styrene copolymer, etc., preferably can be used.

As shown in FIG. 3, in the present embodiment, a hemostasis valve 28, a seal member 30, and a plug 32 are arranged in the interior of the catheter hub 14. The hemostasis valve 28 is formed from an elastic member such as rubber or the like, and has a valve portion having a slit formed in a distal end thereof. The seal member 30 is an annular member, which is constituted from a material (e.g., a porous body) that allows flow of gas yet blocks the flow of liquid through the seal member 30.

The plug 32 is formed in a tubular shape and is arranged movably in the axial direction in the interior of the catheter hub 14. When the catheter hub 14 and the connector of the infusion tube are connected, and upon the plug 32 being moved by the connector in the direction of the distal end, the plug 32 penetrates through the hemostasis valve 28 accompanying elastic deformation of the valve portion provided in the hemostasis valve 28. The hemostasis valve 28, the seal member 30, and the plug 32 need not necessarily be provided.

The inner needle 16 is a rigid tubular member that is capable of puncturing the patient's skin. The inner needle 16 is formed to be sufficiently longer than the catheter 12, such that in an initial condition of the catheter assembly 10, the tip 17 projects out from a distal end opening of the catheter 12, and an intermediate location in the longitudinal direction of the inner needle 16 is inserted through the interior of the catheter hub 14. The proximal end side of the inner needle 16 is retained in the interior of the inner needle hub 18. As the constituent material of the inner needle 16, a metal material, for example, such as stainless steel, aluminum or aluminum alloy, titanium or titanium alloy, may be used.

The inner needle hub 18 makes up the proximal end side of the catheter assembly 10. As shown in FIG. 3, the inner needle hub 18 has a hub main body portion 34 and a needle fixing portion 36.

The hub main body portion 34 is a tubular member formed in a narrow elongate shape having a hollow portion of a predetermined volume, and is formed with an appropriate size (thickness, length) to enable the hub main body portion 34 to be gripped and operated easily by the user when using the catheter assembly 10.

A stopper 37 is fixed to a distal end inner side of the hub main body portion 34. The stopper 37 regulates a maximum insertion position of a later-described outer tube 46 in the interior of the inner needle hub 18, together with defining a maximum advancement position of a later-described adjoining tube 48 with respect to the inner needle hub 18. As shown in FIG. 2, the stopper 37 includes a frame member 38 in which an opening is formed, engaging parts 39a, 39b provided on the left and right side pieces of the frame member 38, and an extending piece 40 which is extended in a distal end direction from an upper side portion of the frame member 38.

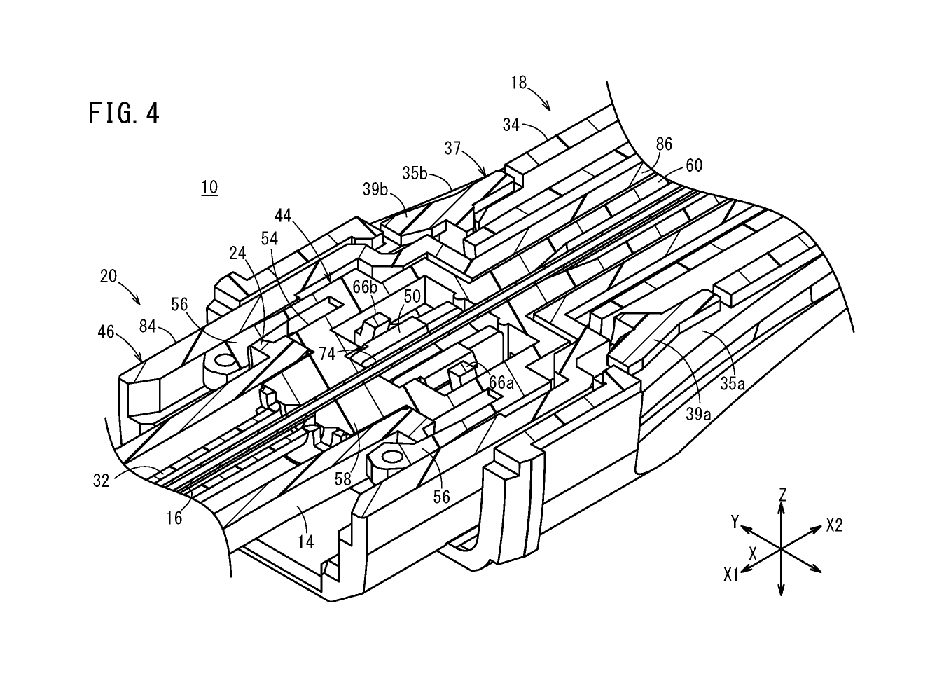

A bent portion 40a, which is bent upwardly, is provided on a distal end part of the extending piece 40. As shown in FIG. 3, the bent portion 40a is arranged in a cutout portion 34a provided on a distal end part of the hub main body portion 34. As shown in FIG. 4, side holes 35a, 35b (see FIG. 2), which are formed on the left and right side walls of the distal end part of the hub main body portion 34, engage respectively with the engaging parts 39a, 39b of the stopper 37, whereby the stopper 37 is fixed in the inner needle hub 18.

As shown in FIG. 3, the needle fixing portion 36 is formed integrally in the interior of the proximal end side of the hub main body portion 34, and fixedly retains the proximal end part of the inner needle 16. A filter 42, which is made of a material that blocks the flow of liquid yet allows air to pass therethrough, is arranged inside the proximal end surface of the needle fixing portion 36. By the proximal end side of the needle fixing portion 36 being closed by the filter 42, a flashback chamber 43 is formed in the interior of the needle fixing portion 36.

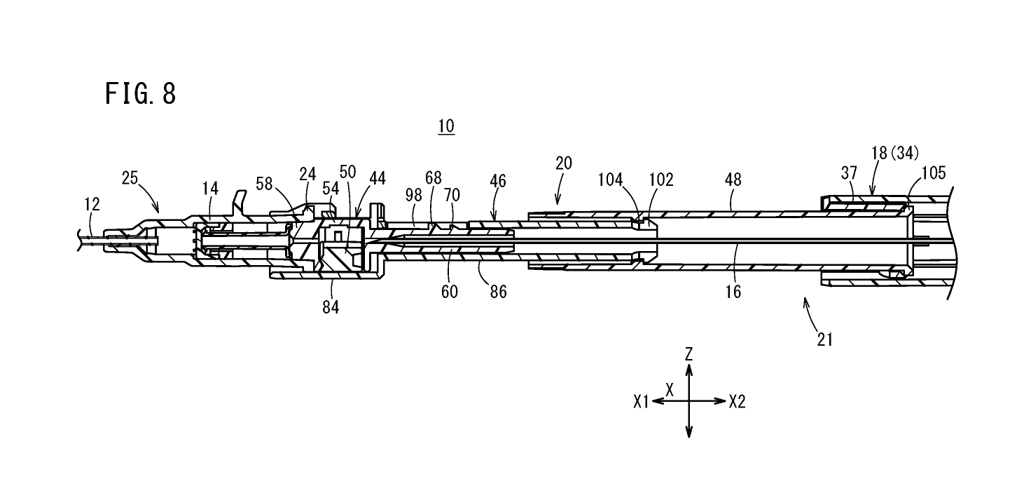

When the inner needle 16 is withdrawn from the catheter 12, the needle protective cover 20 covers the tip 17 of the inner needle 16 as a result of the inner needle 16 being accommodated in the needle protective cover 20. As shown in FIG. 2, the needle protective cover 20 includes an inner tube 44, the outer tube 46, the adjoining tube 48, and a block body 50. When a withdrawal operation of the inner needle 16 from the catheter 12 is carried out, the needle protective cover 20 is extended to cover the entire length of the inner needle 16 (see FIG. 8).

The inner tube 44 is connected detachably to the proximal end of the catheter hub 14, and serves to cover the tip 17 of the inner needle 16 accompanying withdrawal of the inner needle 16 from the catheter 12.

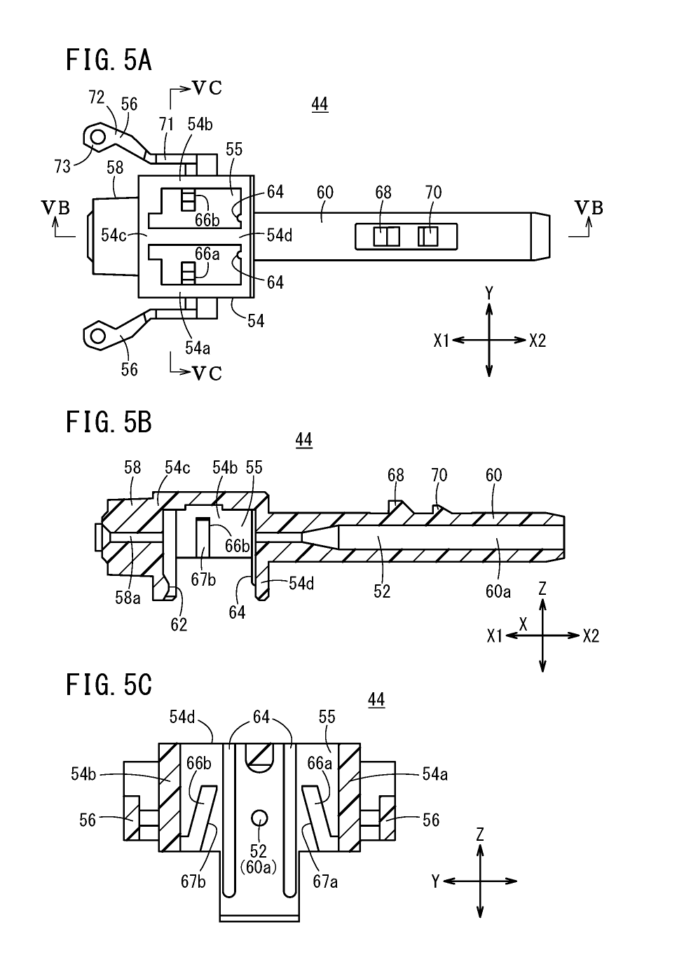

As shown in FIG. 5A, the inner tube 44 includes a block body accommodating section 54 in which the block body 50 is accommodated, a plurality of arms 56 provided integrally on outer sides of the block body accommodating section 54, a distal end tubular section 58 that projects from and is contiguous to the distal end side of the block body accommodating section 54, and a proximal end tubular section 60 that projects from and is contiguous to the proximal end side of the block body accommodating section 54. Further, as shown in FIG. 5B, an inner needle passage 52, which penetrates in the axial direction and in which the inner needle 16 can be inserted, is provided in the inner tube 44.

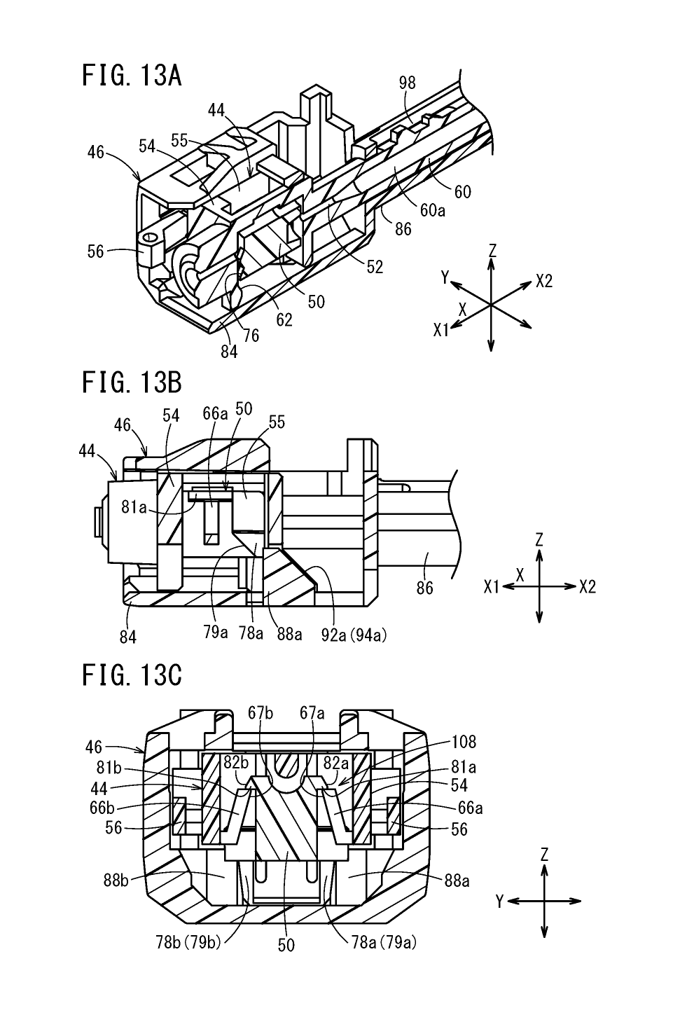

The block body accommodating section 54 accommodates the block body 50 displaceably therein from the non-shielding position (see FIG. 3) where the inner needle passage 52 is not shielded to the shielding position (see FIG. 13A) where the inner needle passage 52 is shielded. In the present embodiment, the block body 50 arrives at the shielding position by rising upwardly from the initial position. An interior space 55 of the block body accommodating section 54 constitutes a portion of the inner needle passage 52 in which the inner needle 16 is insertable.

As shown in FIG. 5B, on an underside rear surface of a front wall 54c of the block body accommodating section 54, an engagement projection 62 is formed that bulges in the direction of the proximal end. Two outwardly bulging and vertically extending guide ribs 64 are formed on a front surface of a rear wall 54d of the block body accommodating section 54. By the guide ribs 64, movement of the block body 50 from the initial position to the shielding position can be stabilized.

Further, on inner surfaces of left and right side walls 54a, 54b of the block body accommodating section 54, latching tabs 66a, 66b (latching members) are formed integrally, which prevent the block body 50 from returning to its initial position by engagement with the block body 50 after movement thereof to the shielding position. The latching tabs 66a, 66b extend in a direction (upwardly in the present embodiment) from the initial position toward the shielding position of the block body 50.

In addition, the latching tabs 66a, 66b are inclined from the supported end sides to the free end sides thereof, so as to approach an inner side of the inner tube 44 (in the present embodiment, a central side in the lateral direction). On the latching tabs 66a, 66b, on portions thereof that undergo sliding contact when the block body 50 is displaced from the initial position to the shielding position, inclined guides 67a, 67b are formed, which transition toward the inner side of the inner tube 44 as the inclined guides 67a, 67b are positioned from the supported end sides toward the free end sides of the latching tabs 66a, 66b.

In FIGS. 5A and 5B, the distal end tubular section 58 is cylindrically shaped, and is fitted into the proximal end of the catheter hub 14, in a state in which the catheter hub 14 and the inner tube 44 are engaged (connected). An interior space 58a of the distal end tubular section 58 constitutes a portion of the inner needle passage 52 in which the inner needle 16 is insertable.

The proximal end tubular section 60 is an elongate pipe-shaped body, which is longer in comparison to the distal end tubular section 58. An interior space 60a of the proximal end tubular section 60 constitutes a portion of the inner needle passage 52 in which the inner needle 16 is insertable. The interior space 58a of the distal end tubular section 58 and the interior space 60a of the proximal end tubular section 60 are disposed on a straight line, and communicate with each other through the interior space 55.

A first protrusion 68 and a second protrusion 70, which are spaced from one another in the axial direction, are provided on an outer circumferential part of the proximal end tubular section 60. A projecting height of the second protrusion 70 is lower than a projecting height of the first protrusion 68.

As shown in FIG. 5A, the arms 56 are capable of engaging releasably from the outside on the proximal end of the catheter hub 14. In the present embodiment, the arms 56 are provided as a pair on left and right side surfaces of the block body accommodating section 54. More specifically, the respective arms 56 have arm base sections 71 that extend in the axial direction from the left and right side surfaces of the block body accommodating portion 54, and engaging end portions 72 connected to distal end sides of the arm base sections 71. Engagement pawls 73, which are capable of engagement with the flange 24 of the catheter hub 14, are disposed integrally on distal end inner sides of the engaging end portions 72.

In a natural state in which no external force is applied, the engaging end portions 72 are inclined so as to spread outwardly toward the direction of the distal end. By connecting locations between the engaging end portions 72 and the arm base sections 71 being elastically deformed, the engaging end portions 72 are configured to be displaceable in directions perpendicular to the axis of the inner tube 44. Operations of the arms 56 will be described in the explanation of the relationship between the inner tube 44 and the outer tube 46.

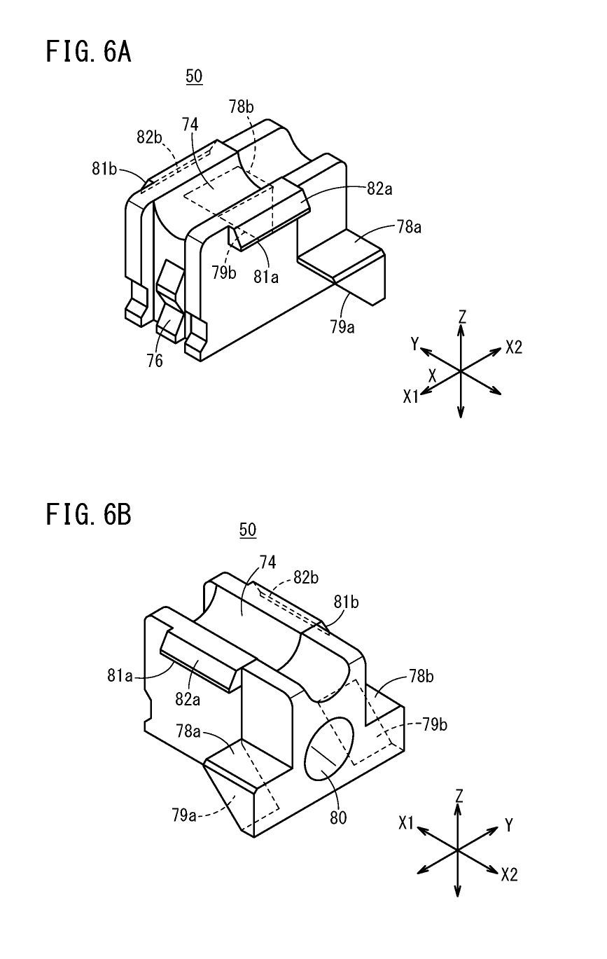

As shown in FIGS. 6A and 6B, a groove 74 extending in forward and rearward directions is formed on an upper part of the block body 50. As shown in FIGS. 3 and 4, in an initial state of the catheter assembly 10, the inner needle 16 is positioned in the groove 74. Further, as shown in FIG. 6A, an engagement recess 76 is formed near a lower portion of the distal end part of the block body 50. In a state with the block body 50 placed in the initial position, the engagement recess 76 engages with the engagement projection 62 (FIG. 5B) provided on the inner tube 44. Consequently, the block body 50 remains fixed in the initial position, and friction between the block body 50 and the inner needle 16 is reduced.

As shown in FIG. 6B, on proximal end lower portions on left and right side surfaces of the block body 50, protrusions 78a, 78b are formed that project laterally outward. The respective protrusions 78a, 78b include inclined surfaces 79a, 79b (second inclined surfaces) inclined with respect to the longitudinal direction (axial direction). More specifically, the inclined surfaces 79a, 79b are inclined so as to transition upwardly toward the distal end direction. A bottomed hole 80 that opens in the proximal end direction is disposed on the proximal end part of the block body 50. In a state in which the block body 50 is placed in the shielding position, the hole 80 faces toward the interior space 60a of the proximal end tubular section 60 of the inner tube 44 (see FIG. 13A).

Further, as shown in FIG. 6B, outwardly (leftward and rightward) projecting pawls 81a, 81b (latched members) are formed on upper sides on the lateral side surfaces of the block body 50. In the illustrated example, the pawls 81a, 81b extend in the longitudinal direction (X direction). The pawls 81a, 81b have inclined surfaces 82a, 82b that transition centrally in the lateral direction as they are positioned upwardly.

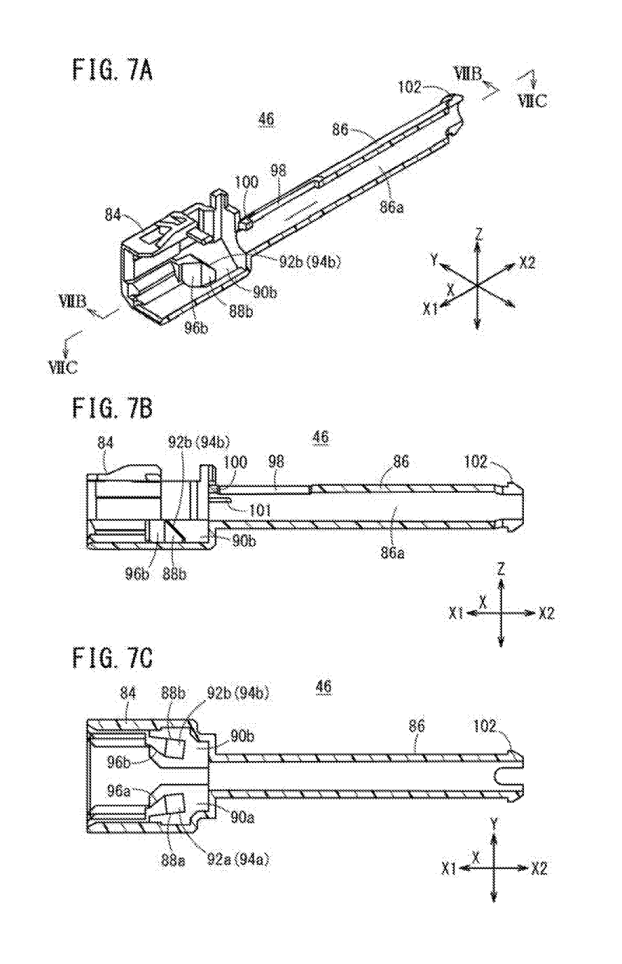

As shown in FIGS. 4 and 7A to 7C, the outer tube 46 includes an arm accommodating section 84 in which the arms 56 can be accommodated, and a tubular section 86 that projects out from the proximal end side of the arm accommodating section 84. FIG. 7A is a perspective view showing in cross section the outer tube 46. FIG. 7B is a cross-sectional view taken along line VIIB-VIIB of FIG. 7A. FIG. 7C is a cross-sectional view taken along line VIIC-VIIC of FIG. 7A.

The arm accommodating section 84 is formed in a box-like shape and opens on the upper part and a distal end part thereof. In the initial state of the catheter assembly 10, the proximal end of the catheter hub 14 and the distal end side of the inner tube 44 (the pair of arms 56 and the block body accommodating section 54) are arranged in the interior of the arm accommodating section 84.

Elastic pieces 88a, 88b which are capable of being deformed elastically in the lateral direction are disposed on a lower part of the arm accommodating section 84. In the case of the present embodiment, left and right openings 90a, 90b that penetrate inside and outside of the arm accommodating section 84 are formed on the lower proximal end side of the arm accommodating portion 84, and the pair of elastic pieces 88a, 88b protrude in the proximal end direction into the left and right openings 90a, 90b.

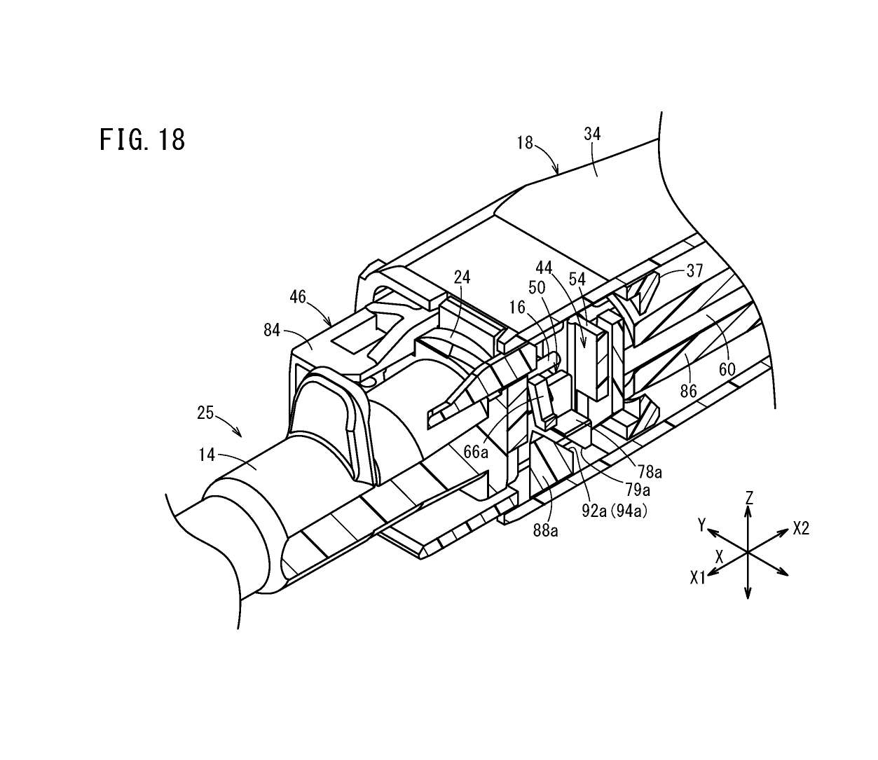

At free end portions (end portions in the X2 direction) of the elastic pieces 88a, 88b, abutting members 92a, 92b are provided, which press the block body 50 toward the shielding position (upwardly in the present embodiment) when an operation of pulling out the inner needle 16 is performed, and accompanying a backward movement of the outer tube 46 with respect to the inner tube 44. In the case of the present embodiment, the abutting members 92a, 92b are inclined surfaces 94a, 94b (first inclined surfaces) which are inclined with respect to the axial direction. In a state with the block body 50 placed in the initial position, the inclined surfaces 94a, 94b of the abutting members 92a, 92b and the inclined surfaces 79a, 79b of the block body 50 face toward each other mutually in parallel (see FIG. 18).

As shown in FIG. 7B, the inclined surfaces 94a, 94b are inclined so as to transition upwardly toward the distal end direction. The angle of the inclined surfaces 94a, 94b with respect to the axial direction is set to from 20 to 80 degrees, and preferably, from 30 to 50 degrees.

As shown in FIGS. 7A and 7C, on inner side surfaces of the supported end sides (sides in the X1 direction) of the elastic pieces 88a, 88b, inclined guides 96a, 96b are provided, which are inclined so as to transition toward the center in the lateral direction as they are positioned in the proximal end direction. In an assembly process of the catheter assembly 10, when the inner tube 44 which is accommodated in the block body 50 is inserted into the outer tube 46, the inclined guides 96a, 96b perform an operation to make it easier for the block body 50 to push out the elastic pieces 88a, 88b toward the outer side. The angle of the inclined guides 96a, 96b with respect to the axial direction is set to from 5 to 75 degrees, and preferably, from 15 to 45 degrees.

As shown in FIG. 7C, according to the present embodiment, the pair of elastic pieces 88a, 88b are inclined slightly inwardly in the lateral direction and do not extend mutually in parallel. According to this constitution, when performing an operation to pull out the inner needle 16, it is difficult for the elastic pieces 88a, 88b to become deformed in an outward direction, and it is possible to effectively prevent the elastic pieces 88a, 88b from being pushed out by the block body 50. However, if desired, the pair of elastic pieces 88a, 88b may extend mutually in parallel with each other.

In the tubular section 86 of the outer tube 46, a lumen 86a that communicates with the interior of the arm accommodating section 84 is formed to penetrate in the axial direction. On an upper part at a location near the distal end of the tubular section 86, an elongate slit 98, which penetrates through the inside and outside of the tubular section 86, is disposed along the axial direction of the tubular section 86.

An engagement tab 100, which is elastically deformable in a diametrical direction of the outer tube 46, is provided on a distal end upper part of the tubular section 86. In the illustrated example, the engagement tab 100 is formed between two small slits 101 provided on a distal end part of the outer tube 46, and which are separated in the circumferential direction. A hook 102, which projects outwardly and extends in a circumferential direction, is provided on the outer side surface of the proximal end part of the outer tube 46.

As shown in FIGS. 2 and 3, the adjoining tube 48 includes a lumen 48a in which the tubular section 86 of the outer tube 46 can be accommodated, and is assembled in a slidable manner relatively with respect to the outer tube 46. A hook 104, which projects inwardly and extends in a circumferential direction, is provided on an inner side surface near the distal end of the adjoining tube 48. The hook 104 is capable of engaging with the hook 102 provided on the outer tube 46. A hook 105, which projects outwardly and extends in a circumferential direction, is provided on the outer side surface of the proximal end part of the adjoining tube 48. The hook 105 is capable of engaging with the stopper 37 that is fixed on an inner side of the hub main body portion 34.

The materials constituting the aforementioned respective members (the inner tube 44, the block body 50, the outer tube 46, the adjoining tube 48) of the inner needle hub 18 and the needle protective cover 20 are not particularly limited, and for example, may be the same materials as cited in the description of the catheter hub 14. In this case, all of these members may be formed from the same material, or may be formed from different materials for each of the members.

The catheter assembly 10 according to the present embodiment is constituted basically as described above. Below, operations and advantages of the catheter assembly 10 will be described.

As shown in FIG. 1, in the initial condition of the catheter assembly 10, the inner needle 16 is inserted into the catheter 12, and the tip 17 protrudes out by a predetermined length from the distal end of the catheter 12. As shown in FIG. 3, the distal end tubular section 58 of the inner tube 44 is inserted into the proximal end of the catheter hub 14, and the outer tube 46 is moved maximally within the movable range thereof toward the distal end side with respect to the inner tube 44.

Further, as shown in FIG. 4, in the initial condition of the catheter assembly 10, the pair of arms 56 provided on the inner tube 44 are positioned inside the arm accommodating section 84 of the outer tube 46, whereby the arms 56 are placed in a closed state. The closed pair of arms 56 engage with the flange 24 of the catheter hub 14, whereby separation of the catheter hub 14 from the needle protective cover 20 including the inner tube 44 is prevented.

Furthermore, as shown in FIG. 3, in the initial condition of the catheter assembly 10, the inner needle 16 penetrates through the interior space 55 of the block body accommodating section 54 of the inner tube 44, and rising of the block body 50, which is positioned at the initial position, is prevented by the inner needle 16. Consequently, displacement of the outer tube 46 toward the proximal end direction with respect to the inner tube 44 is prevented by the block body 50. Further, the adjoining tube 48 is inserted maximally into the inner needle hub 18, and the tubular section 86 of the outer tube 46 is inserted maximally into the adjoining tube 48. In this state, the arm accommodating section 84 is inserted into the distal end side of the inner needle hub 18.

After the protector 22 has been removed (see FIG. 1), a user (a doctor or medical technician, etc.) performs an operation to grip the inner needle hub 18 of the catheter assembly 10, whereby the catheter 12 and the inner needle 16 puncture and are inserted into a blood vessel of a patient. After puncturing, a detachment operation (withdrawal operation of the inner needle 16) is carried out in order to detach the coupling body (referred to below as an “inner needle unit 21”), which is made up from the inner needle 16, the inner needle hub 18, and the needle protective cover 20, from the catheter member 25.

In the detachment operation, in a state in which the position of the catheter member 25 is maintained, the inner needle hub 18 is moved and retracted in the proximal end direction. Upon doing so, the inner needle 16, which is retained inside the inner needle hub 18, starts to be retracted with respect to the catheter 12. On the other hand, a condition (movement stopped state) is brought about, in which the needle protective cover 20 cannot be displaced with respect to the catheter member 25 until the inner needle 16 has been retracted by a predetermined amount.

When the inner needle hub 18 is retracted by a predetermined amount, since the hook 105 of the proximal end side of the adjoining tube 48 engages with the stopper 37 that is fixed to the distal end side of the inner needle hub 18, accompanying retraction of the inner needle hub 18, the adjoining tube 48 also is retracted. When the inner needle hub 18 is further retracted, the hook 104 disposed on the inner side of the adjoining tube 48 engages with the hook 102 on the proximal end side of the tubular section 86 of the outer tube 46.

Consequently, as shown in FIG. 8, a condition is brought about in which the outer tube 4 6, the adjoining tube 48, and the inner needle hub 18 are expanded to the maximum extent. Further, in this state, together with the inner needle hub 18 being retracted with respect to the adjoining tube 48, since by retraction of the adjoining tube 48 with respect to the outer tube 46, the needle protective cover 20 also is expanded. Thus, the inner needle 16 is covered over its entire length by the inner needle hub 18 and the needle protective cover 20.

During the process of retracting the inner needle hub 18 with respect to the catheter member 25 as described above, the inner needle 16 also is retracted with respect to the inner tube 44. At this time, as shown in FIG. 8, when the tip 17 of the inner needle 16 moves more toward the proximal end side than the block body 50 that is arranged inside the inner tube 44, a state is brought about in which the block body 50 can be moved from the initial position to the blocking position. Nonetheless, at the point in time shown by FIG. 8, the arms 56 provided on the inner tube 44 still remain closed, as in the state shown in FIG. 4, and engagement between the inner tube 44 and the catheter hub 14 is maintained.

As a result of the state being brought about in which the block body 50 can move from its initial position to the blocking position, displacement of the outer tube 46 toward the proximal end side with respect to the inner tube 44 becomes possible. Consequently, from the condition in which the outer tube 46, the adjoining tube 48, and the inner needle hub 18 are displaced relatively and expanded maximally in the axial direction, when a movement operation is performed to move the inner needle hub 18 further in the proximal end direction, the outer tube 46 is displaced in the proximal end direction with respect to the inner tube 44.

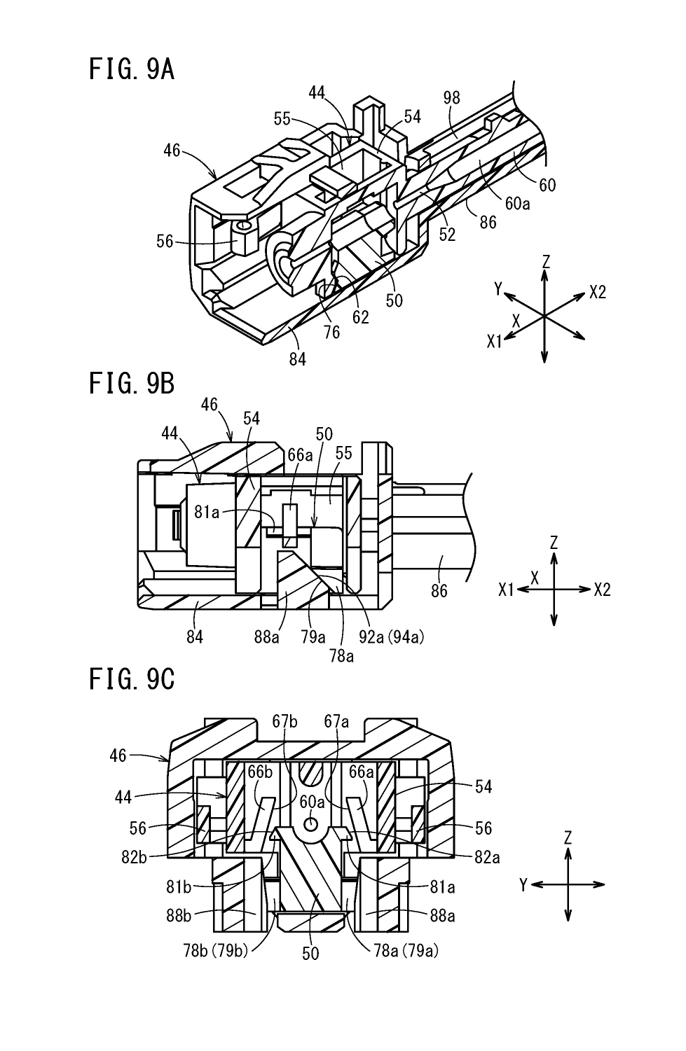

Next, a description will be made with reference to FIGS. 9A to 13C of a mutual positional relationship between the inner tube 44, the outer tube 46, and the block body 50, in which accompanying displacement of the outer tube 46 in the proximal end direction with respect to the inner tube 44, the block body 50 undergoes displacement from the initial position to the shielding position. However, in FIGS. 9A to 13C, illustration of the catheter hub 14 and the inner needle 16 is omitted.

In FIGS. 9A through 13C, drawings which share the same FIG. numbers (for example, FIGS. 9A to 9C) are cross-sectional views of different locations at the same point in time, and in particular, A is a perspective cross-sectional view at a position where the inner needle passage 52 is visible, B is a cross-sectional view at a position where the relationship between the abutting members 92a, 92b (inclined surfaces 94a, 94b) of the outer tube 46 and the block body 50 is visible, and C is a cross-sectional view at a position where the relationship between the latching tabs 66a, 66b and the block body 50 is visible.

As shown in FIGS. 9A to 9C, when displacement of the outer tube 46 in the proximal end direction with respect to the inner tube 44 is started, the block body 50 begins to rise upwardly. More specifically, as shown in FIG. 9B, the abutting members 92a, 92b (inclined surfaces 94a, 94b) provided on the outer tube 46 come into contact with the inclined surfaces 79a, 79b provided on the block body 50, and accompanying relative displacement in the axial direction between the outer tube 46 and the inner tube 44, by a tapering action, the abutting members 92a, 92b start to push the block body 50 upwardly. Moreover, as shown in FIG. 9C, at this point in time, the pawls 81a, 81b of the block body 50 are not in contact with the latching tabs 66a, 66b of the inner tube 44.

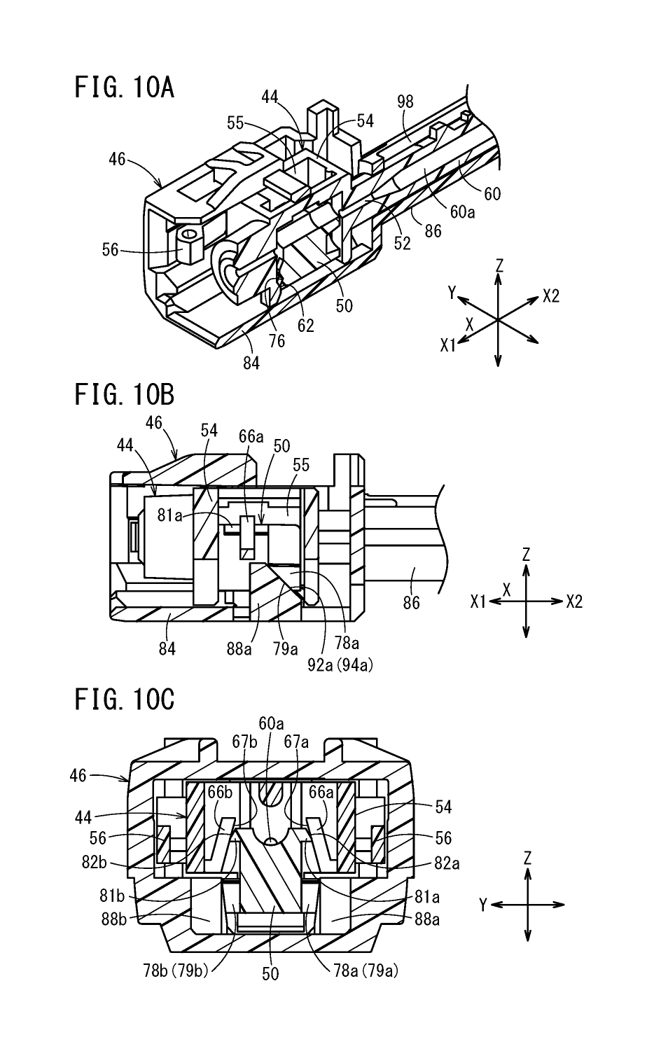

As shown in FIGS. 10A to 10C, when the outer tube 46 is displaced further in the proximal end direction with respect to the inner tube 44, the block body 50 rises upward further. At this time, as shown in FIG. 10C, the pawls 81a, 81b of the block body 50 start to come into contact with the latching tabs 66a, 66b. In this case, since the inclined guides 67a, 67b are provided on inner sides of the latching tabs 66a, 66b, the pawls 81a, 81b slide smoothly with respect to the latching tabs 66a, 66b.

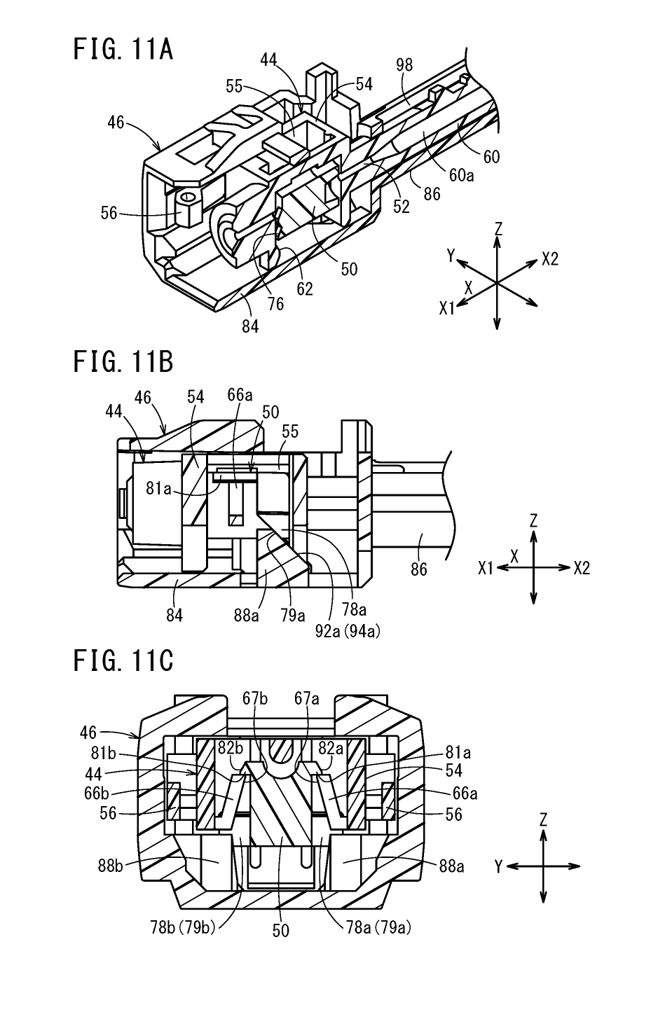

Additionally, as shown in FIGS. 11A to 11C, when the outer tube 46 is displaced further in the proximal end direction with respect to the inner tube 44, the block body 50 rises further upward. In a process from the state shown in FIG. 10C to the state shown in FIG. 11C, the latching tabs 66a, 66b are pressed outward by the pawls 81a, 81b and are elastically deformed, such that when the pawls 81a, 81b overcome the latching tabs 66a, 66b, the latching tabs 66a, 66b are displaced toward the block body 50 by the elastic restoring force thereof. In the case of the present embodiment, as shown in FIG. 11B, at a point in time before the block body 50 has risen maximally, as shown in FIG. 11C, the pawls 81a, 81b of the block body 50 overcome the latching tabs 66a, 66b, and the pawls 81a, 81b come into engagement once with the latching tabs 66a, 66b.

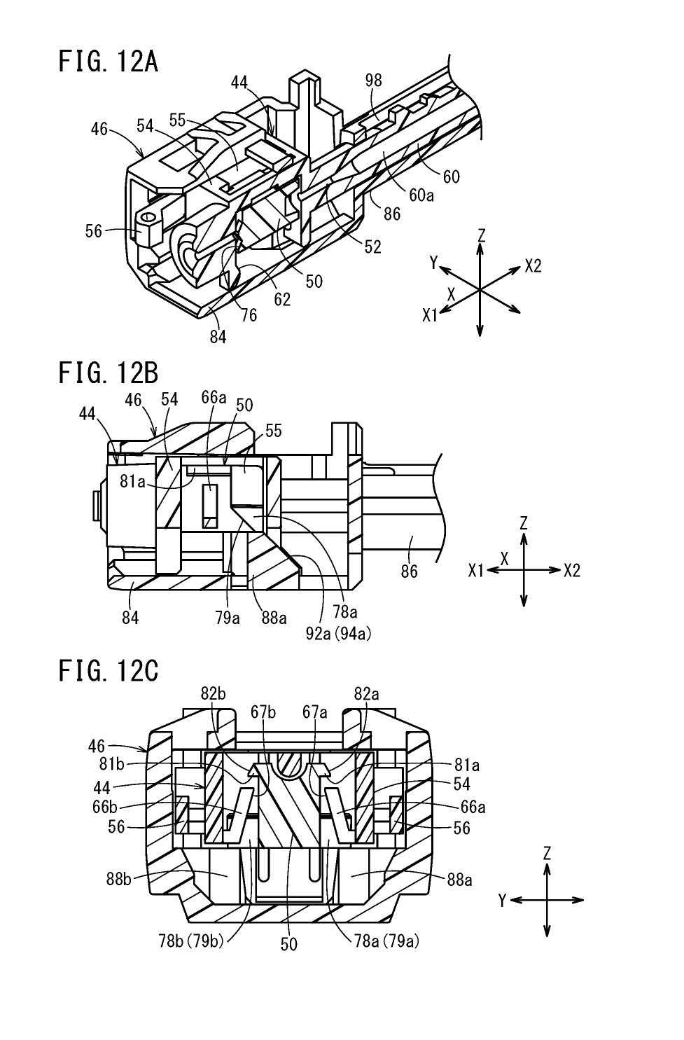

After the pawls 81a, 81b of the block body 50 have overcome the latching tabs 66a, 66b, when the outer tube 46 is displaced further in the proximal end direction with respect to the inner tube 44, as shown in FIGS. 12A to 12C, the block body 50 rises further upward. More specifically, as shown in FIG. 12B, the block body 50 reaches the upper ends of the abutting members 92a, 92b, and as shown in FIG. 12C, the pawls 81a, 81b of the block body 50 become separated temporarily from the latching tabs 66a, 66b.

In addition, after the block body 50 has reached the upper ends of the abutting members 92a, 92b, when the outer tube 46 is displaced further in the proximal end direction with respect to the inner tube 44, the block body 50 descends slightly and stops. More specifically, as shown in FIG. 13B, the block body 50 overcomes the abutting members 92a, 92b and falls back downward. At this time, as shown in FIG. 13C, the pawls 81a, 81b of the block body 50 come into contact and engage with the latching tabs 66a, 66b.

In this manner, since the block body 50 ends in a state of shielding the inner needle passage 52 accompanying relative displacement between the inner tube 44 and the outer tube 46 in the axial direction, the tip 17 of the inner needle 16 is prevented from projecting out again from the distal end of the inner tube 44.

Further, in a state in which the block body 50 has reached the shielding position, by the pawls 81a, 81b of the block body 50 being engaged with the latching tabs 66a, 66b provided on the inner tube 44, downward descent of the block body 50 is prevented, and the block body 50 is reliably maintained in a state of shielding the inner needle passage 52. In this manner, a lock mechanism 108 is constructed in which the latching tabs 66a, 66b provided on the inner tube 44 and the pawls 81a, 81b provided on the block body 50 fix the condition in which the block body 50 shields the inner needle passage 52 (the block body 50 is restrained in the shielding position).

In addition, when the outer tube 46 is displaced further in the proximal end direction with respect to the inner tube 44 from the state shown in FIGS. 13A to 13C, the engaging end portions 72 of the arms 56 project out in the distal end direction from the arm accommodating section 84. Consequently, since the restriction on expansion applied to the arms 56 by the arm accommodating section 84 is released, the arms 56 are actively displaced (widened) outwardly by the elastic restoring forces thereof.

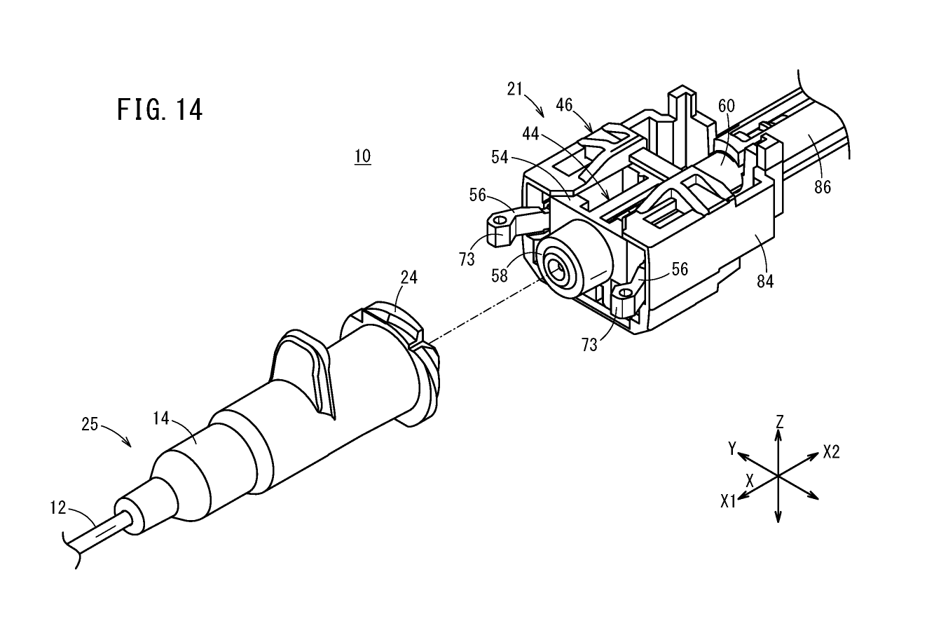

Upon doing so, as shown in FIG. 14, engagement between the arms 56 provided on the inner tube 44 and the flange 24 provided on the catheter hub 14 is released, and therefore, accompanying movement of the inner needle hub 18 in the proximal end direction, the inner tube 44 separates from the catheter hub 14. Stated otherwise, the inner needle unit 21 is separated from the catheter member 25. Moreover, at this point in time at which the inner needle unit 21 has been separated from the catheter member 25, the entire length of the inner needle 16 is already accommodated in the interior of the needle protective cover 20 and the inner needle hub 18, and a condition is brought about in which the tip 17 of the inner needle 16 is covered.

After the inner needle 16 has been withdrawn from the catheter 12, a non-illustrated connector of an infusion tube is connected to the proximal end side of the catheter hub 14, whereby supply of an infusion agent (medicinal solution) is carried out from the infusion tube to the patient.

As described above, according to the catheter assembly 10 of the present embodiment, when an operation to withdraw the inner needle 16 is performed, the block body 50, which is arranged in the interior of the inner tube 44, is pressed by the abutting members 92a, 92b provided in the outer tube 46, whereby the block body 50 moves from the initial position to the shielding position (see FIGS. 9A to 13C). Thus, it is possible to shield the inner needle passage 52, and to prevent the inner needle 16 from protruding to the exterior of the inner tube 44.

Further, since the state in which the block body 50 shields the inner needle passage 52 is maintained by the lock mechanism 108 (see FIG. 13C), protrusion of the inner needle 16 to the exterior can more effectively be prevented.

Furthermore, due to the fact that the latching members (latching tabs 66a, 66b) of the lock mechanism 108 are configured to be elastically displaceable inside the inner tube 44, they are easily deformed when the latched members (pawls 81a, 81b of the block body 50) overcome the latching members (latching tabs 66a, 66b). Therefore, an increase in the operating force required to perform the withdrawal operation of the inner needle 16, which is caused by resistance to activation of the lock mechanism 108, can effectively be suppressed.

In the case of the present embodiment, the latching members that are engaged with the pawls 81a, 81b of the block body 50 are the latching tabs 66a, 66b, which are supported in a cantilevered fashion by the inner surface of the inner tube 44 (see FIG. 13C). In accordance with this configuration, since the latching members are easily deformed, resistance to activation of the lock mechanism 108 can be effectively suppressed.

In the case of the present embodiment, the latching tabs 66a, 66b extend in a direction from the initial position toward the shielding position of the block body 50 (see FIG. 13C). In accordance with this configuration, since the latching tabs 66a, 66b are easily deformed accompanying movement of the block body 50, resistance to activation of the lock mechanism 108 can more effectively be suppressed.

In the case of the present embodiment, on the latching tabs 66a, 66b, on portions thereof that undergo sliding contact when the block body 50 is displaced from the initial position to the shielding position, the inclined guides 67a, 67b are formed, which deviate to or are inclined to the inner side of the inner tube 44 as the inclined guides 67a, 67b are positioned from the supported end sides toward the free end sides of the latching tabs 66a, 66b (see FIG. 13C). In accordance with this configuration, under a guiding action of the inclined guides 67a, 67b, displacement takes place smoothly between the block body 50 and the latching tabs 66a, 66b, and resistance to activation of the lock mechanism 108 can more effectively be suppressed.

In the case of the present embodiment, in a state with the block body 50 placed in the initial position, the inclined surfaces 94a, 94b of the abutting members 92a, 92b and the inclined surfaces 79a, 79b of the block body 50 face toward each other in parallel (see FIG. 18). In accordance with this configuration, when the abutting members 92a, 92b press the block body 50, because the inclined surfaces contact and slide against each other, the block body 50 can be made to move stably and smoothly.

In the case of the present embodiment, the inclined surfaces 79a, 79b of the block body 50 and the inclined surfaces 94a, 94b of the abutting members 92a, 92b are arranged in plurality, respectively, while being separated in a widthwise direction of the block body 50 (see FIGS. 6A and 7C). In accordance with this configuration, movement of the block body 50 can be realized in a more stable manner.

In the case of the present embodiment, since the abutting members 92a, 92b are formed on the elastic pieces 88a, 88b provided on the outer tube 46 (see FIG. 7C), in the assembly process for the catheter assembly 10, when the inner tube 44 is inserted into the outer tube 46, the elastic pieces 88a, 88b are pressed and deformed elastically by the block body 50. Thus, the inner tube 44 can be smoothly inserted into the outer tube 46 without causing damage to the inner tube 44 and the outer tube 46. Details concerning this feature of the present embodiment will be discussed later.

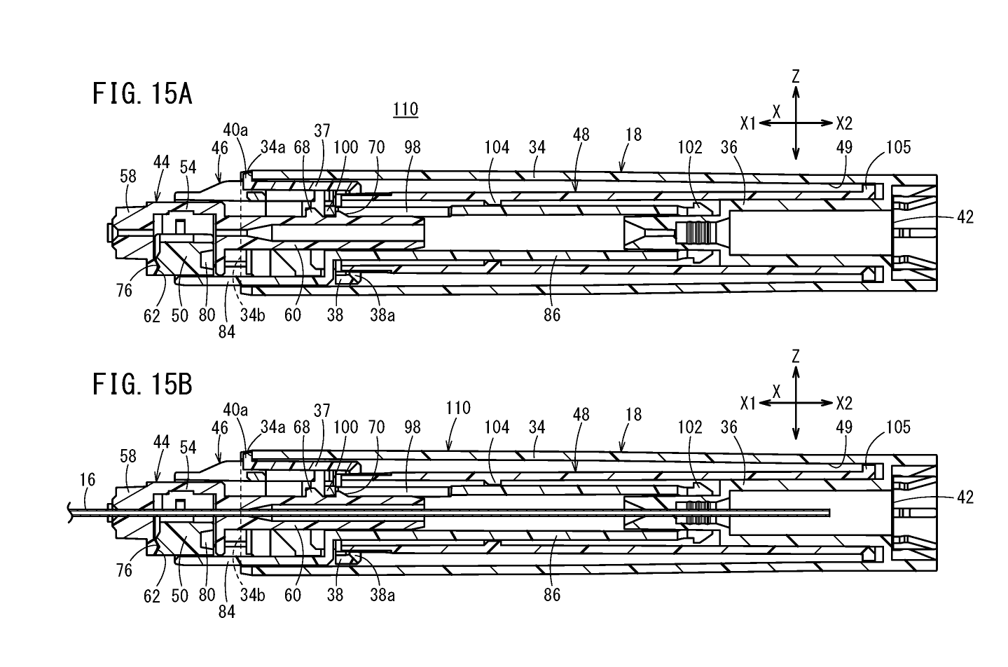

Next, primarily with reference to FIGS. 15A to 18, a manufacturing method (assembly method) for the aforementioned catheter assembly 10 will be described.

First, a description will be given of an assembly process for a cover and hub assembly 110 shown in FIG. 15A. The cover and hub assembly 110 is a semi-manufactured product of the catheter assembly 10, and the catheter assembly 10 is completed by assembly of the inner needle 16 and the catheter member 25 together with the cover and hub assembly 110.

In the assembly process of the cover and hub assembly 110, at first, the adjoining tube 48 is inserted into the inner needle hub 18 through a distal end opening 34b of the inner needle hub 18 (adjoining tube insertion step). In this case, a proximal end part of the adjoining tube 48 is arranged on a proximal end side of an annular groove 49 formed between the hub main body portion 34 and the needle fixing portion 36 in the inner needle hub 18, together with the needle fixing portion 36 being inserted into the adjoining tube 48.

Next, the stopper 37 is attached to an inner side of the inner needle hub 18 through the distal end opening 34b of the inner needle hub 18 (stopper attachment step). In this case, the stopper 37 is inserted inside the inner needle hub 18 while the engaging parts 39a, 39b are pressed and elastically deformed inwardly, and when the engaging parts 39a, 39b (see FIG. 2) arrive at the side holes 35a, 35b (see FIG. 2) of the inner needle hub 18, they are deformed outwardly by the elastic restoring force thereof and are inserted into the side holes 35a, 35b, and become engaged with the side holes 35a, 35b. As a result, the stopper 37 is fixed and retained on an inner side in the vicinity of the distal end part of the inner needle hub 18.

Next, the outer tube 46 is inserted into the adjoining tube 48 through the distal end opening 34b of the inner needle hub 18 (outer tube insertion step). In this case, the tubular section 86 of the outer tube 46 is inserted through an opening 38a of the stopper 37, and the needle fixing portion 36 of the inner needle hub 18 is inserted into the tubular section 86. By engagement of a proximal end part of the arm accommodating section 84 with the frame member 38 of the stopper 37, the outer tube 46 stops upon being inserted up to a predetermined position of the inner needle hub 18.

Next, the inner tube 44 in a state with the block body 50 arranged at the initial position (hereinafter also referred to an “assembled inner tube”) is inserted into the outer tube 46 up to a predetermined preliminary fixing position (inner tube preliminary insertion step). In this instance, in a process of obtaining the assembled inner tube (block body arranging step), the block body 50 is inserted into the block body accommodating section 54 from below the inner tube 44, and the block body 50 is arranged at the initial position. At this time, the engagement recess 76 provided in the block body 50 (see FIG. 6A) engages with the engagement projection 62 (see FIG. 5B) provided in the inner tube 44 with a weak engaging force, whereby the block body 50 is prevented from shifting unintentionally from the initial position, and falling out from the inner tube 44 and entering into the shielding position. The block body arranging step may be implemented before the outer tube insertion step or in parallel with the outer tube insertion step.

In the inner tube preliminary insertion step, during the process of inserting the inner tube 44 into the outer tube 46 up to the preliminary fixing position, the engagement tab 100 of the outer tube 46 is elastically deformed outwardly, whereby the second protrusion 70 overcomes the engagement tab 100 and is capable of moving toward the proximal end side from the engagement tab 100. By arranging the engagement tab 100 provided on the outer tube 46 between the first protrusion 68 and the second protrusion 70 provided on the inner tube 44, the inner tube 44 is positioned with respect to the outer tube 46 in the preliminary fixing position.

With the cover and hub assembly 110 shown in FIG. 15A, the inner tube 44 is not inserted with respect to the outer tube 46 up to the insertion completion position, and the engaging end portions 72 (see FIG. 5A) of the arms 56 provided on the inner tube 44 project out from the distal end side of the arm accommodating section 84 of the outer tube 46. Therefore, a condition is brought about in which the engaging end portions 72 are inclined in outward directions with respect to the arm base sections 71, or stated otherwise, the arms 56 are in an opened state.

The cover and hub assembly 110 which is assembled in the foregoing manner can be maintained in this state for a certain period of time while being transported or stored, etc., and until it is combined together with the inner needle 16 and the catheter member 25 to bring about completion thereof as the catheter assembly 10. Moreover, the protector 22 (see FIG. 1) may be mounted on the distal end part of the cover and hub assembly 110, until the process of combining the cover and hub assembly 110 together with the inner needle 16 and the catheter member 25 is carried out.

In the assembly process of the aforementioned cover and hub assembly 110, since the adjoining tube 48, the stopper 37, the outer tube 46, and the inner tube 44 (the inner tube 44 assembled together with the block body 50) are assembled sequentially in one direction with respect to the inner needle hub 18 from the distal end side toward the proximal end side of the inner needle hub 18, ease of assembly can be enhanced. Further, since it is possible to adopt an assembly method in which components thereof can be assembled by dropping other components with reference to the inner needle hub 18, automated assembly by a robot can be easily realized.

Next, the process of assembling the inner needle 16 and the catheter member 25 together with the cover and hub assembly 110 and completion of the catheter assembly 10 will be described.

As shown in FIG. 15B, the inner needle 16 is inserted into the cover and hub assembly 110, and the proximal end side of the inner needle 16 is fixed in the needle fixing portion 36. In this case, the means for fixing the inner needle 16 with respect to the needle fixing portion 36 is not particularly limited. For example, the proximal end side of the inner needle 16 and the needle fixing portion 36 may be adhered and fixed to each other by applying an ultraviolet curable adhesive to a predetermined location of the needle fixing portion 36, and irradiating the applied ultraviolet curable adhesive with ultraviolet rays to cure the adhesive.

Next, the filter 42 is fixed, for example, by welding or adhesion, to an inside proximal end part of the needle fixing portion 36.

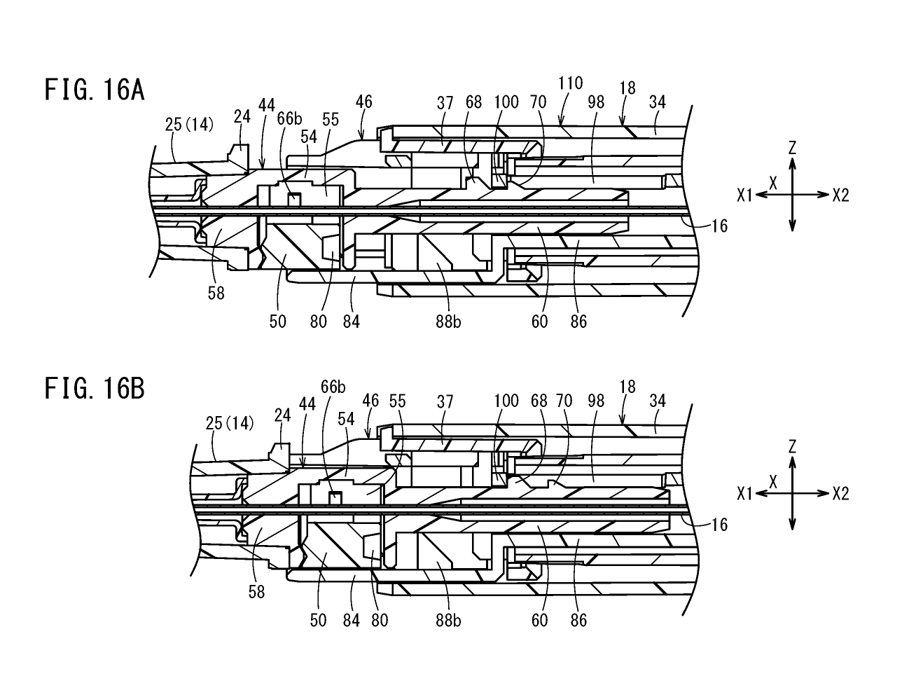

Next, as shown in FIG. 16A, the catheter member 25 is mounted on a distal end part of the cover and hub assembly 110. More specifically, together with the inner needle 16 being inserted through the catheter 12, the distal end tubular section 58 of the inner tube 44 is fitted into the proximal end part of the catheter hub 14. Moreover, at this point in time, the inner tube 44 still remains in the preliminary fixing position of the outer tube 46, and the arms 56 provided on the inner tube 44 are in an opened state.

Next, as shown in FIG. 16B, when the catheter member 25 is pressed inward in the proximal end direction with respect to the inner needle hub 18, the first protrusion 68 provided on the inner tube 44 overcomes the engagement tab 100 provided on the outer tube 46 and enters into the slit 98. In this case, by outward (upward) elastic deformation and displacement of the engagement tab 100, the first protrusion 68 is capable of overcoming the engagement tab 100.

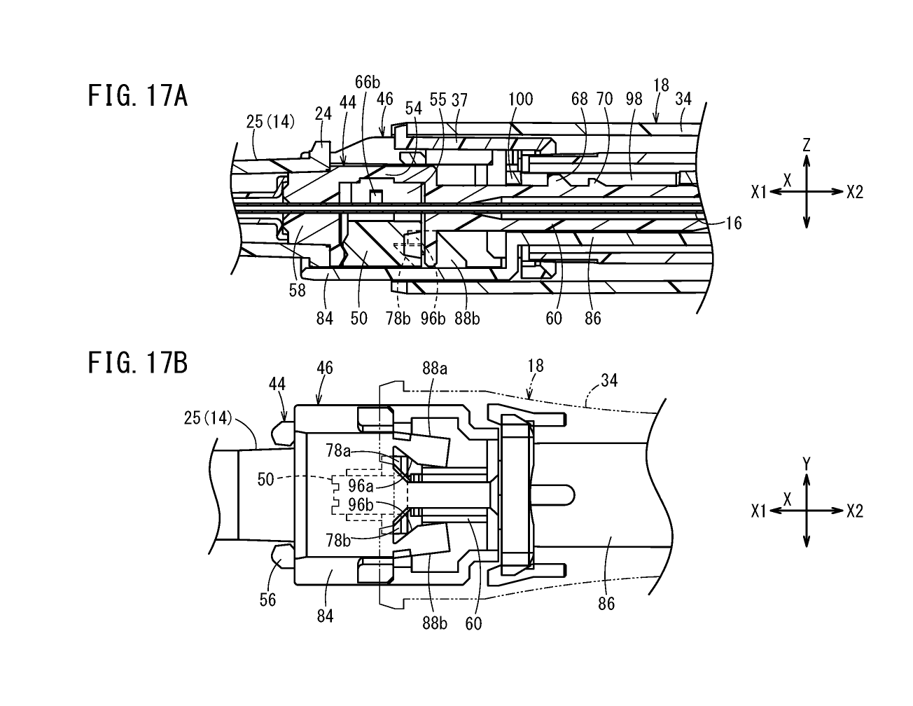

In addition, when the catheter member 25 and the inner tube 44 are pressed further inward in the proximal end direction with respect to the inner needle hub 18, as shown in FIGS. 17A and 17B, the protrusions 78a, 78b provided on the block body 50 that is accommodated in the inner tube 44 come into contact with the elastic pieces 88a, 88b provided on the outer tube 46. In this case, the protrusions 78a, 78b come into contact with the inclined guides 96a, 96b formed on the inner sides of the elastic pieces 88a, 88b. FIG. 17B is a view showing the inner tube 44, the outer tube 46, and the block body 50 of FIG. 17A, as viewed from a bottom surface side.

From the state shown in FIGS. 17A and 17B, when the catheter member 25 and the inner tube 44 are pressed further inward in the proximal end direction with respect to the inner needle hub 18, the block body 50 moves in the direction of the proximal end while pressing and elastically deforming the elastic pieces 88a, 88b outwardly (in the Y direction). In this case, since the protrusions 78a, 78b of the block body 50 slide in contact with the inclined guides 96a, 96b, the block body 50 is capable of moving smoothly. Upon movement of the block body 50 to a predetermined position, the protrusions 78a, 78b of the block body 50 overcome the elastic pieces 88a, 88b. When the protrusions 78a, 78b of the block body 50 overcome the elastic pieces 88a, 88b, the elastic pieces 88a, 88b are displaced inwardly (to the side of the block body 50) by the elastic restoring force thereof.

In the foregoing manner, since the protrusions 78a, 78b of the block body 50 overcome the elastic pieces 88a, 88b by elastically deforming the elastic pieces 88a, 88b, the inner tube 44 can be smoothly inserted into the outer tube 46 without the inner tube 44 and the outer tube 46 becoming damaged.

FIG. 18 is a perspective cross-sectional view showing a state after the protrusions 78a, 78b of the block body 50 have overcome the elastic pieces 88a, 88b, the catheter member 25 is pressed further inward in the proximal end direction with respect to the inner needle hub 18. In this case, the inner tube 44 arrives at a position (insertion completion position) maximally on the proximal end side with respect to the outer tube 46. Further, the abutting members 92a, 92b (inclined surfaces 94a, 94b) and the inclined surfaces 79a, 79b of the block body 50 face toward each other in parallel through a slight gap.

Incidentally, during the process in which the inner tube 44 moves in the proximal end direction with respect to the outer tube 46, the arms 56 provided on the inner tube 44 are displaced inwardly as they become accommodated in the arm accommodating section 84, and a closed state is brought about. Consequently, the flange 24 provided at the proximal end of the catheter hub 14 and the engaging end portions 72 of the arms 56 engage with each other, and separation of the catheter hub 14 and the inner tube 44 is prevented.

Upon completion of the manufacturing method (assembly step), the catheter assembly 10 in the condition shown in FIG. 1 is completed.

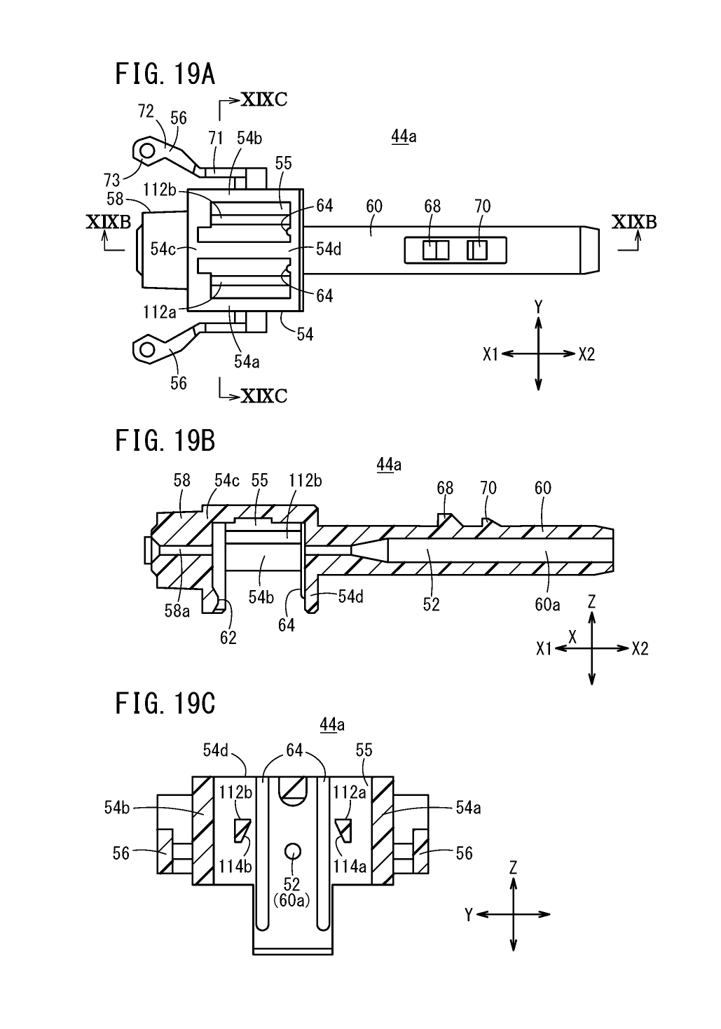

With the inner tube 44 of the above-described catheter assembly 10, although the latching tabs 66a, 66b are provided as latching members that are engaged with the pawls 81a, 81b of the block body 50, instead of the latching tabs 66a, 66b, beams 112a, 112b may be provided, as in an inner tube 44a shown in FIGS. 19A to 19C.

With the inner tube 44a of the illustrated example, two beams 112a, 112b are disposed mutually in parallel at a given interval in the lateral direction (Y direction). Further, the beams 112a, 112b extend along the axial direction (X direction), and both ends thereof are supported by the front wall 54c and the rear wall 54d of the block body accommodating section 54 of the inner tube 44a. In this manner, the beams 112a, 112b are capable of being deformed elastically in the lateral direction (Y direction) between both ends in the direction of extension thereof. Moreover, the beams 112a, 112b are formed integrally with the inner tube 44a.

As shown in FIG. 19C, on inner sides of the beams 112a, 112b, inclined guides 114a, 114b are provided that transition inwardly (centrally in the lateral direction) as they are positioned upwardly.

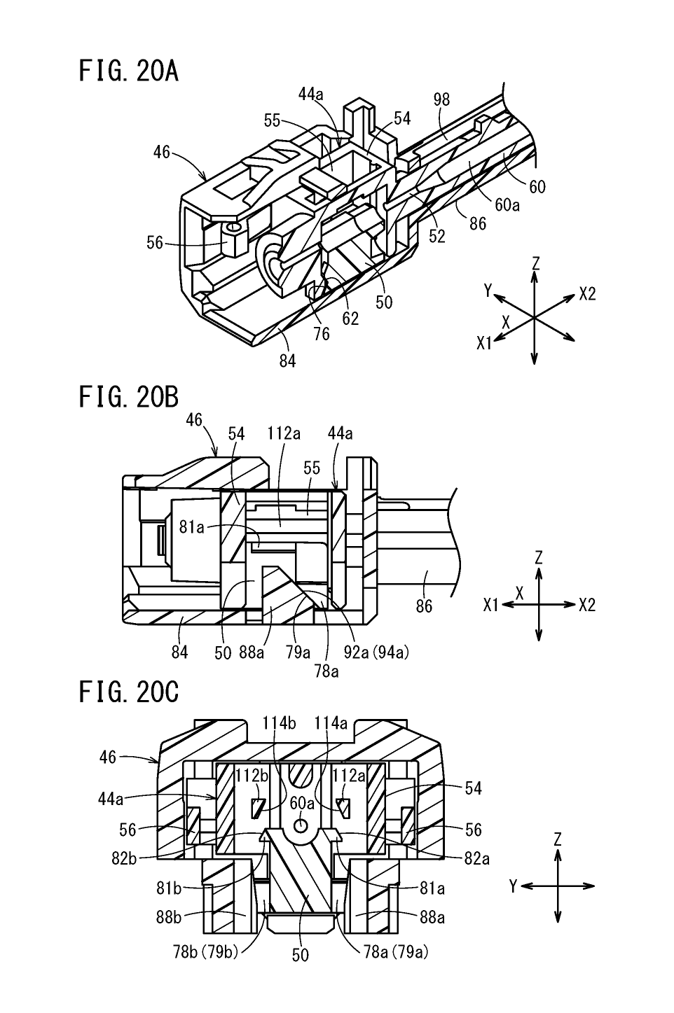

Next, operations during use of the catheter assembly 10 in which the inner tube 44a is employed will be described, focusing on a mutual positional relationship between the inner tube 44a, the outer tube 46, and the block body 50. In this instance, a description will be made with reference to FIGS. 20A to 24C of the mutual positional relationship between the inner tube 44a, the outer tube 46, and the block body 50, in which accompanying displacement of the outer tube 46 in the proximal end direction with respect to the inner tube 44a, the block body 50 undergoes displacement from the initial position to the shielding position. However, in FIGS. 20A to 24C, illustration of the catheter hub 14 and the inner needle 16 is omitted.

Moreover, in the catheter assembly 10 in which the inner tube 44a having the beams 112a, 112b is adopted, operations until the needle protective cover 20 is extended maximally when the inner needle 16 is withdrawn (operations until reaching the state of FIG. 8) are the same as those of the catheter assembly 10 in which the inner tube 44 having the latching tabs 66a, 66b is adopted.

As shown in FIGS. 20A to 20C, accompanying withdrawal of the inner needle 16, when displacement of the outer tube 46 in the proximal end direction with respect to the inner tube 44a is started, the block body 50 begins to rise upwardly. More specifically, as shown in FIG. 20B, the abutting members 92a, 92b (inclined surfaces 94a, 94b) provided on the outer tube 46 come into contact with the inclined surfaces 79a, 79b provided on the block body 50, and accompanying relative displacement in the axial direction between the outer tube 46 and the inner tube 44a, by a tapering action, the abutting members 92a, 92b start to push the block body 50 upwardly. Moreover, as shown in FIG. 20C, at this point in time, the pawls 81a, 81b of the block body 50 are not in contact with the beams 112a, 112b of the inner tube 44a.

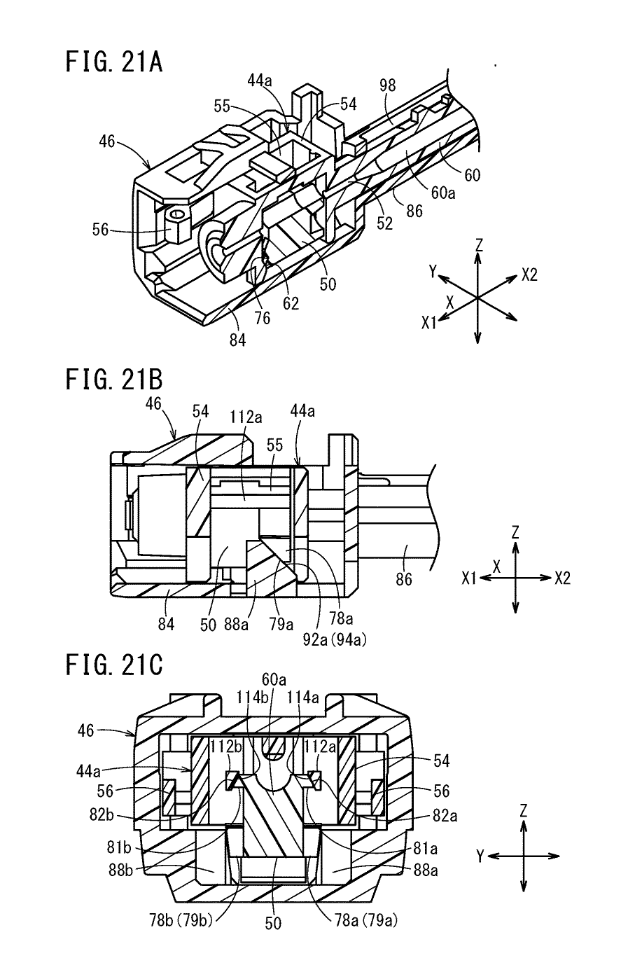

As shown in FIGS. 21A to 21C, when the outer tube 46 is displaced further in the proximal end direction with respect to the inner tube 44a, the block body 50 rises upward further. At this time, as shown in FIG. 21C, the pawls 81a, 81b of the block body 50 start to come into contact with the beams 112a, 112b. In this case, since the inclined guides 114a, 114b are provided on inner sides of the beams 112a, 112b, the pawls 81a, 81b slide smoothly with respect to the beams 112a, 112b.

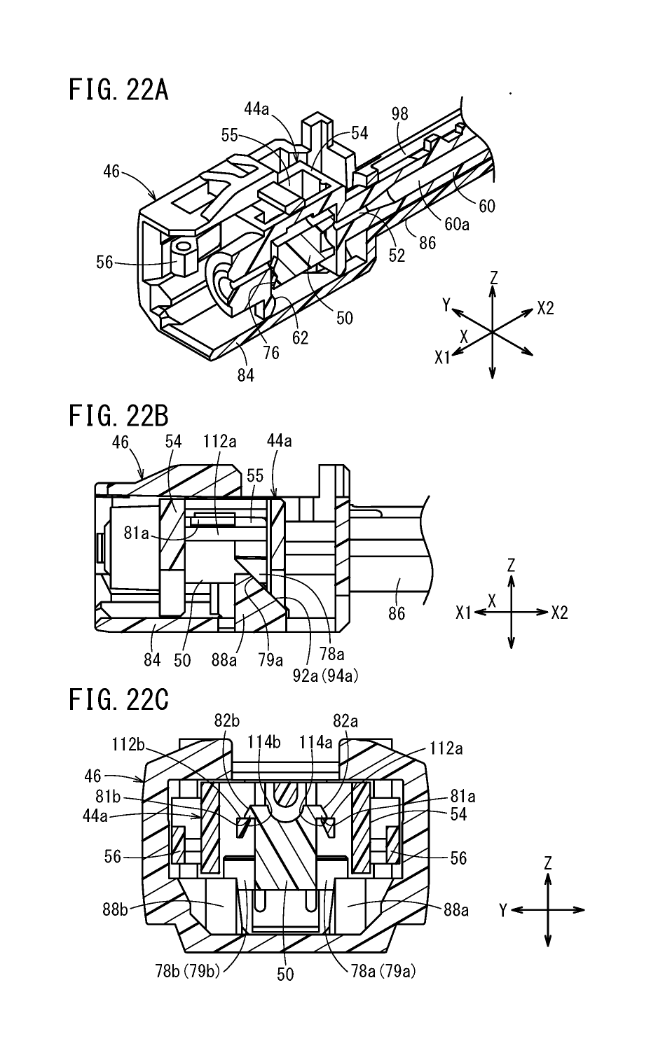

In addition, as shown in FIGS. 22A to 22C, when the outer tube 46 is displaced further in the proximal end direction with respect to the inner tube 44a, the block body 50 rises upward further. In a process from the state shown in FIG. 21C to the state shown in FIG. 22C, the beams 112a, 112b are pressed outward by the pawls 81a, 81b and are elastically deformed, such that when the pawls 81a, 81b overcome the beams 112a, 112b, the beams 112a, 112b are displaced toward the block body 50 by the elastic restoring force thereof. In the case of the present embodiment, as shown in FIG. 22B, at a point in time before the block body 50 has risen maximally, as shown in FIG. 22C, the pawls 81a, 81b of the block body 50 overcome the beams 112a, 112b, and the pawls 81a, 81b come into engagement once with the beams 112a, 112b.

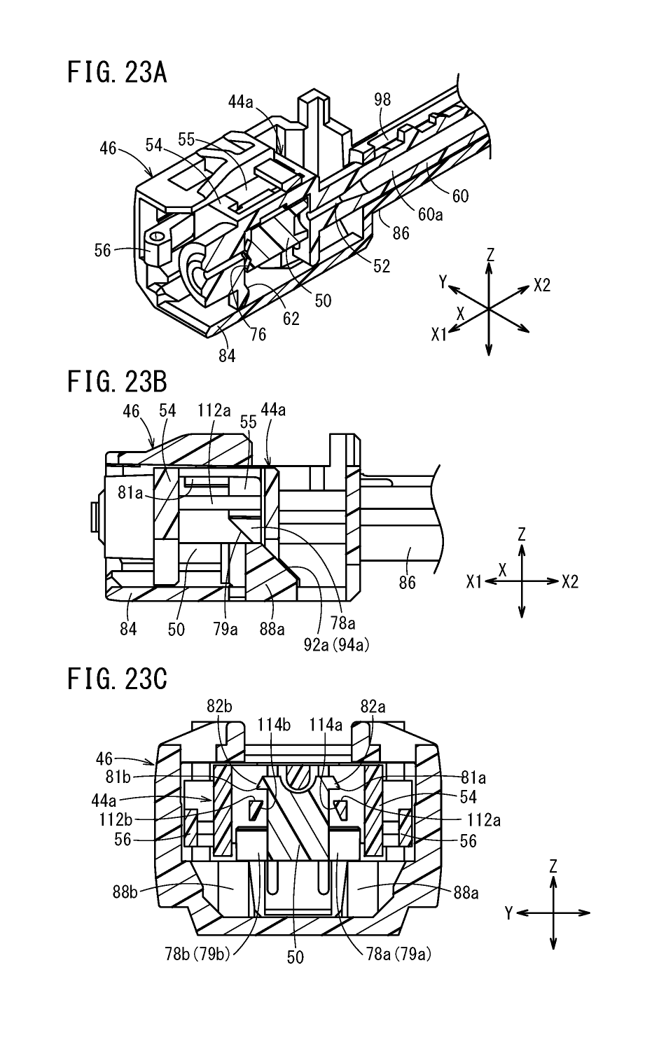

After the pawls 81a, 81b of the block body 50 have overcome the beams 112a, 112b, when the outer tube 46 is displaced further in the proximal end direction with respect to the inner tube 44a, as shown in FIGS. 23A to 23C, the block body 50 rises further upward. More specifically, as shown in FIG. 23B, the block body 50 reaches the upper ends of the abutting members 92a, 92b, and as shown in FIG. 23C, the pawls 81a, 81b of the block body 50 become separated temporarily from the beams 112a, 112b.

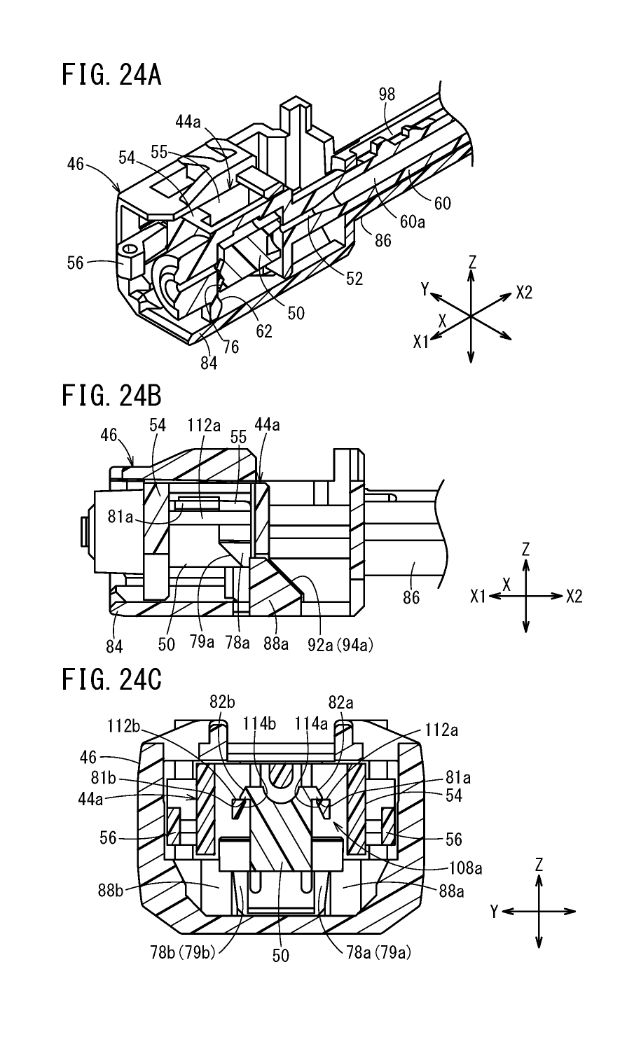

In addition, after the block body 50 has reached the upper ends of the abutting members 92a, 92b, when the outer tube 46 is displaced further in the proximal end direction with respect to the inner tube 44a, as shown in FIGS. 24A to 24C, the block body 50 descends slightly and stops. More specifically, as shown in FIG. 24B, the block body 50 overcomes the abutting members 92a, 92b and falls back downward. At this time, as shown in FIG. 24C, the pawls 81a, 81b of the block body 50 come into contact and engage with the beams 112a, 112b.

In this manner, since the block body 50 ends in a state of shielding the inner needle passage 52 accompanying relative displacement between the inner tube 44a and the outer tube 46 in the axial direction, the tip 17 of the inner needle 16 is prevented from projecting out again from the distal end of the inner tube 44a.

Further, in a state in which the block body 50 has reached the shielding position, by the pawls 81a, 81b of the block body 50 being engaged with the beams 112a, 112b provided on the inner tube 44a, downward descent of the block body 50 is prevented, and the block body 50 is reliably maintained in a state of shielding the inner needle passage 52. In this manner, a lock mechanism 108a is constructed in which the beams 112a, 112b provided on the inner tube 44a and the pawls 81a, 81b provided on the block body 50 fix the condition in which the block body 50 shields the inner needle passage 52 (the block body 50 is restrained in the shielding position).

Moreover, the manufacturing method (assembly method) of the catheter assembly 10 in which the inner tube 44a is adopted is the same as the aforementioned manufacturing method of the catheter assembly 10 in which the inner tube 44 is adopted.

Although a preferred embodiment of the present invention has been described, the present invention is not limited to the above-described embodiment. It goes without saying that various modifications can be adopted therein without departing from the spirit and scope of the invention.

1. A catheter assembly comprising:
an inner needle including a sharp tip at a distal end thereof;
a catheter through which the inner needle is inserted;
a catheter hub connected to a proximal end part of the catheter; and
a needle protective cover configured to cover at least the tip of the inner needle when the inner needle is withdrawn;
the needle protective cover including:
an inner tube including an inner needle passage penetrating in an axial direction, and being detachably connected to the catheter hub;
an outer tube inside of which the inner tube is arranged, and the outer tube configured to be relatively displaceable in the axial direction with respect to the inner tube;
a block body accommodated in the inner tube, and configured to be displaceable from an initial position at which the inner needle passage is not shielded to a shielding position at which the inner needle passage is shielded; and
a lock mechanism configured to restrain the block body at the shielding position;
wherein the lock mechanism includes a latching member provided in the inner tube and configured to be elastically displaceable in interior of the inner tube, and a latched member disposed on the block body and configured to be capable of engagement with the latching member; and
when an operation to withdraw the inner needle is performed, accompanying a backward movement of the outer tube with respect to the inner tube, the block body is configured to be pressed by an abutting member formed in the outer tube to move from the initial position to the shielding position, and the block body is configured to be retained at the shielding position by the lock mechanism.

2. The catheter assembly according to claim 1, wherein the latching member is a latching tab supported in a cantilevered fashion by an inner surface of the inner tube.

3. The catheter assembly according to claim 2, wherein the latching tab is configured to extend in a direction from the initial position toward the shielding position of the block body.

4. The catheter assembly according to claim 3, wherein, on the latching tab, on a portion thereof that undergoes sliding contact with the block body when the block body is displaced from the initial position to the shielding position, an inclined guide is formed, which deviates to an inner side of the inner tube as the inclined guide is positioned from a supported end side toward a free end side of the latching tab.

5. The catheter assembly according to claim 1, wherein the latching member is a beam supported at both ends thereof by inner surfaces of the inner tube.

6. The catheter assembly according to claim 1, wherein:
the abutting member includes a first inclined surface which is inclined with respect to a direction of relative movement between the outer tube and the inner tube; and
the block body includes a second inclined surface which faces toward the first inclined surface in a state of being positioned at the initial position.

7. The catheter assembly according to claim 6, wherein the first inclined surface and the second inclined surface are arranged in plurality, respectively, while being separated in a widthwise direction of the block body.

8. The catheter assembly according to claim 1, wherein:
the block body includes a protrusion which protrudes in the widthwise direction and is pressed by the abutting member;
an elastic piece, which is elastically deformable in the widthwise direction of the block body, is formed on the outer tube; and
the abutting member is formed on the elastic piece.

9. A manufacturing method for a catheter assembly, the catheter assembly comprising:
an inner needle including a sharp tip at a distal end thereof;
a catheter through which the inner needle is inserted;
a catheter hub connected to a proximal end part of the catheter; and
a needle protective cover configured to cover at least the tip of the inner needle when the inner needle is withdrawn;
the needle cover including:
an inner tube including an inner needle passage penetrating in an axial direction, and being detachably connected to the catheter hub;
an outer tube inside of which the inner tube is arranged, and the outer tube configured to be relatively displaceable in the axial direction with respect to the inner tube;
a block body accommodated in the inner tube, an configured to be displaceable from an initial position at which the inner needle passage is not shielded to a shielding position at which the inner needle passage is shielded; and
a lock mechanism configured to restrain the block body at the shielding position;
wherein the lock mechanism includes a latching member provided in the inner tube and configured to be elastically displaceable in interior of the inner tube, and a latched member disposed on the block body and configured to be capable of engagement with the latching member;
when an operation to withdraw the inner needle is performed, accompanying a backward movement of the outer tube with respect to the inner tube, the block body is configured to be pressed by an abutting member formed in the outer tube to move from the initial position to the shielding position, and the block body is configured to be retained at the shielding position by the lock mechanism;
the block body includes a protrusion which protrudes in the widthwise direction and is pressed by the abutting member;
an elastic piece, which is elastically deformable in the widthwise direction of the block body, is formed on the outer tube; and
the abutting member is formed on the elastic piece,
the method comprising:
a block body arranging step of arranging the block body in the initial position inside the inner tube; and
an inner tube insertion step of, after the block body arranging step, inserting the inner tube into the outer tube up to an insertion completion position;
wherein, in the inner tube insertion step, accompanying relative displacement of the block body with respect to the outer tube, the protrusion of the block body presses the elastic piece formed on the outer tube outwardly, and elastically displaces the elastic piece, whereby the protrusion overcomes the elastic piece.

10. The manufacturing method for the catheter assembly according to claim 9, wherein:
the catheter assembly includes an inner needle hub connected to a proximal end part of the inner needle;
the needle protective cover includes an adjoining tube in which the outer tube is slidably inserted, and which is slidably inserted in the inner needle hub;
a needle fixing portion configured to retain the proximal end part of the inner needle is formed integrally in the inner needle hub; and
the catheter assembly comprises a stopper configured to prevent the adjoining tube from being pulled out in a distal end direction from the inner needle hub;
the manufacturing method further comprising:
an adjoining tube insertion step of inserting the adjoining tube into the inner needle hub through a distal end opening of the inner needle hub;
a stopper attachment step of, after the adjoining tube insertion step, attaching the stopper to the inner needle hub through the distal end opening of the inner needle hub;
an outer tube insertion step of, after the stopper attachment step, inserting the outer tube into the adjoining tube through the distal end opening of the inner needle hub; and
an inner tube preliminary insertion step of, after the outer tube insertion step and the block body arranging step, inserting the inner tube into the outer tube up to a predetermined preliminary fixing position, through a distal end opening of the outer tube.
